(19) United States
(12) Patent Application Publication
(10) Pub. No.: US 2016/0168071 A1
(43) Pub. Date: Jun. 16, 2016

(54) PHOSPHORAMIDITE DERIVATIVES IN THE HYDROFORMYLATION OF UNSATURATED COMPOUNDS

(71) Applicant: EVONIK DEGUSSA GMBH, Essen (DE)

(72) Inventors: Katrin Marie DYBALLA, Recklinghausen (DE); Robert FRANKE, Marl (DE); Dirk FRIDAG, Haltern am See (DE); Eduard BENETSKIY, Ryazan (RU); Armin BÖRNER, Rostock (DE); Susan LÜHR, Santiago (CL); Detlef SELENT, Rostock (DE)

(73) Assignee: EVONIK DEGUSSA GMBH, Essen (DE)

(21) Appl. No.: 14/906,671

(22) PCT Filed: Jul. 22, 2014
(86) PCT No.: PCT/EP2014/065704

§ 371 Date: Jan. 21, 2016

(30) Foreign Application Priority Data
Jul. 23, 2013 (DE) 10 2013 214 373.7

(51) Int. Cl. C07C 67/38; C07F 15/00; C07F 9/6571; B01J 31/18; B01J 31/02

(52) U.S. Cl. CPC C07C 67/38; B01J 31/186; B01J 31/0255; C07F 9/657154; C07F 15/0073; B01J 2231/321; B01J 2531/822

(57) ABSTRACT

The invention relates to the following subject matters: a) phosphoramidites of the formula (I); b) transition metal-containing compounds of the formula Me(acac)(CO)L, wherein L is selected from the formula (I); c) catalytically active compositions in the hydroformylation, which contain the compounds listed in a) and b); d) method for hydroformylation of unsaturated compounds using the catalytically active composition listed under c); and e) multi-phase reaction mixture containing unsaturated compounds, gas mixture comprising carbon monoxide, hydrogen, aldehydes, and the catalytically active compositions described in c).

In terms of volume, hydroformylation is one of the most important homogeneous catalyses on the industrial scale. The aldehydes obtained thereby are important intermediates or end products in the chemical industry (Rhodium Catalyzed Hydroformylation, P. W. N. M. van Leeuwen, C. Claver, eds.; Kluver Academic Publishers: Dordrecht Netherlands; 2000. R. Franke, D. Selent, A. Börmer, Chem. Rev. 2012, 112, 5675). Hydroformylation with Rh catalysts is of particular significance.

In addition to the hydroformylation of unfunctionalized olefins, reaction with functionalized substrates, including those olefins which are obtained from renewable raw materials in particular, is gaining significance. In this context, the hydroformylation of unsaturated fatty acids plays a major role (A. Behr, Fat. Sci. Technol. 1990, 92, 375-388. A. Behr, A. Westfechtel, Chem. Ing. Tech. 2007, 79, 621-636. A. Behr, A. Westfechtel, J. Perez Gomes, Chem. Eng. Technol. 2008, 31, 700-714).

For control of activity and regioselectivity of the catalyst, usually trivalent phosphorus compounds are used as organic ligands. Particularly phosphites, i.e. compounds containing P—O bonds, have become very widely used for this purpose (EP 0054986; EP 0697391; EP 213639; EP 214622; U.S. Pat. No. 4,769,498; DE 10031493; DE 102006058682; WO 2008124468).

Phosphoramidites, i.e. compounds having one or more P—N bonds rather than the P—O bonds, have to date been used only rarely as ligands in hydroformylation.

Van Leeuwen and coworkers (A. van Rooy, D. Burgers, P. C. J. Kamer, P. W. N. M. van Leeuwen, Recl. Trav. Chim. Pays-Bas 1996, 115, 492) were the first to study monodentate phosphoramidites in hydroformylation. Overall, only moderate catalytic properties were observed at the high to extremely high ligand/rhodium ratios of up to 1000:1. At the lowest ligand/rhodium ratio, or P/Rh ratio, of 10:1, a high isomerization activity and the formation of non-hydroformylated internal olefins was found. Only increasing the P/Rh ratio increased the TOF to a moderate 910 h⁻¹ and enhanced the selectivity.

The use of chiral phosphoramidites for asymmetric catalyses was claimed in WO 2007/031065, without giving working examples specifically for asymmetric hydroformylation. Chiral bidentate ligands each having a phosphoramidite unit have been used in various forms in asymmetric hydroformylation (J. Mazuela, O. Pàmies, M. Diéguez, L. Palais, S. Rosset, A. Alexakis, Tetrahedron: Asymmetry 2010, 21, 2153-2157; Y. Yan, X. Zhang, J. Am. Chem. Soc. 2006, 128, 7198-7202; Z. Hua, V. C. Vassar, H. Choi, I. Ojima, PNAS 2004, 13, 5411-5416).

Of paramount importance for the efficacy of the catalyst is the stability of the ligand towards various chemical agents before, during and after the catalysis (the latter in the case of intentional recycling). One of the main causes of the breakdown of phosphite ligands, which, unlike phosphines, are very stable towards oxygen, is the reaction with water, which leads to cleavage of the P—O bonds (Homogeneous Catalysts, Activity-Stability-Deactivation, P. W. N. M. van Leeuwen, J. C. Chadwick, eds.; Wiley-VCH, 2011, p. 23 ff.). The hydrolysis gives rise particularly to pentavalent phosphorus compounds which have lost most of their ligand properties. Water forms almost unavoidably under almost all hydroformylation conditions through aldol condensation of the product aldehydes. Furthermore, water is a constant companion of functionalized olefins which are obtained from vegetable raw materials.

In general, a greater tendency to react with nucleophiles (e.g. water or alcohols) is attributed to phosphoramidites than phosphites. This property is utilized widely, for example, for the synthesis of phosphites from phosphoramidites (e-EROS Encyclopedia of Reagents for Organic Synthesis. doi:10.1002/047084289X.rn00312; R. Hulst, N. K. de Vries, B. L. Feringa, Tetrahedron: Asymmetry 1994, 5, 699-708), but at the same time raises particular questions about the suitability thereof as ligands of long-term stability for catalysis.

The use of suitable phosphorus substituents can contribute to the stabilization of phosphorus compounds at risk of hydrolysis. The only method described to date in the context of phosphoramidite ligands is the use of N-pyrrolyl radicals on the phosphorus (WO 02/083695). Substituents on the heterocycle, for example 2-ethylpyrrolyl (WO 03018192, DE 102005061642) or indolyl (WO 03/018192), improve hydrolysis stability still further.

The hydrolytic breakdown of phosphoramidite ligands can also be slowed by the addition of amines to the hydroformylation reaction, as taught in EP 1677911, US 2006/0224000 and U.S. Pat. No. 8,110,709.

The use of hydrolysis-stable pyrrolylphosphines or the addition of basic stabilizers greatly narrows the scope of application of the hydroformylation reaction to these working examples.

It is an object of the present invention to provide hydrolysis-stable ligands for catalytically active compositions for chemical synthesis of organic compounds, especially the hydroformylation, the hydrocyanation and the hydrogenation of unsaturated compounds. As well as the ease of synthesis of the phosphoramidites and the use thereof as ligands, a high yield of product and a high n/i selectivity are to be achieved in the hydroformylation.

The object is achieved by phosphoramidites of the formula (I):

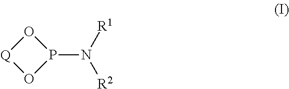

Unexpectedly, small lactam rings in particular impart extremely high hydrolysis stability to the phosphoramidite. This hydrolysis stability was confirmed by extended ³¹P NMR analyses.

The present invention provides phosphoramidites of the formula (I) where Q is a divalent substituted or unsubstituted aromatic radical;

where R¹ is not the same as R² and they are independently selected from alkyl, aryl, carboxamide and organosulphonyl radicals;
or R¹ and R² together with N form a heterocyclic structure selected from lactams, dicarboximides.

Preferably, Q is selected from substituted or unsubstituted 1,1′-biphenyl, 1,1′-binaphthyl and ortho-phenyl radicals.

More preferably, Q is selected from substituted or unsubstituted 1,1′-biphenyl radicals.

Preferred substituted 1,1′-biphenyl radicals are those having, in the 3,3′ and/or 5,5′ positions in the 1,1′-biphenyl-2,2′-diol base skeleton, an alkyl radical and/or an alkoxy radical, preferably a C₁-C₄-alkyl radical, more preferably a tert-butyl radical (t-Bu) and/or preferably a C₁-C₅-alkoxy radical, more preferably a methoxy radical.

It is advantageous when R¹ is not the same as R² and they are independently selected from C₁-C₁₀-alkyl, preferably C₁-C₅-alkyl, aryl, carboxamide and tosyl radicals.

Particularly preferred compounds of the formula (I) are selected from:

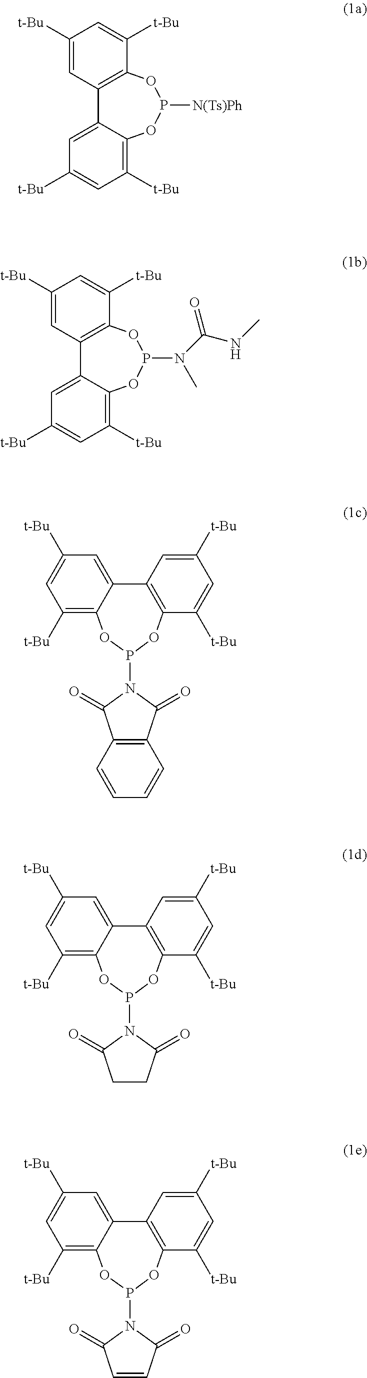

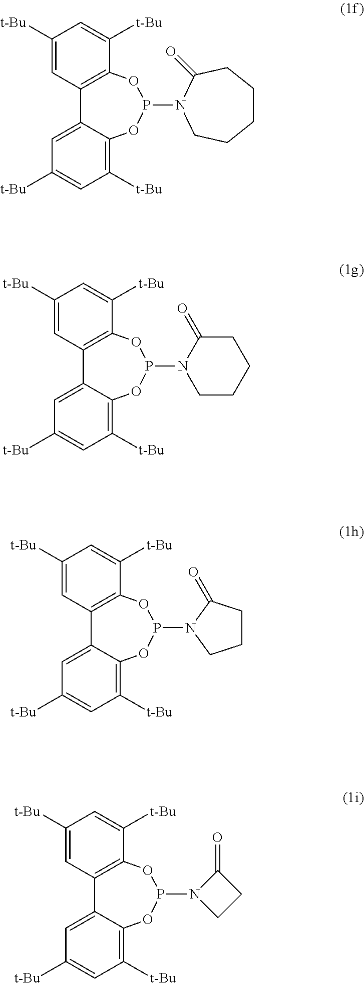 [SMILES: CC(C)(C)c1cc(C(C)(C)C)c2op(N3CCCCCC3=O)oc3c(C(C)(C)C)cc(C(C)(C)C)cc3c2c1 ; CC(C)(C)c1cc(C(C)(C)C)c2op(N3CCCCC3=O)oc3c(C(C)(C)C)cc(C(C)(C)C)cc3c2c1 ; CC(C)(C)c1cc(C(C)(C)C)c2op(N3CCCC3=O)oc3c(C(C)(C)C)cc(C(C)(C)C)cc3c2c1 ; CC(C)(C)c1cc(C(C)(C)C)c2op(N3CCC3=O)oc3c(C(C)(C)C)cc(C(C)(C)C)cc3c2c1]

The present invention further provides transition metal compounds of the formula Me(acac)(CO)L with Me=transition metal, where L is selected from:

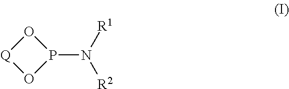

where Q is a divalent substituted or unsubstituted aromatic radical;
where R¹ is not the same as R² and they are independently selected from alkyl, aryl, carboxamide and organosulphonyl radicals;
or R¹ and R² together with N form a heterocyclic structure selected from lactams, dicarboximides.

Preferably, Q is selected from substituted or unsubstituted 1,1′-biphenyl, 1,1′-binaphthyl and ortho-phenyl radicals.

More preferably, Q is selected from substituted and unsubstituted 1,1′-biphenyl radicals.

Preferred substituted 1,1′-biphenyl radicals are those having, in the 3,3′ and/or 5,5′ positions in the 1,1′-biphenyl-2,2′-diol base skeleton, an alkyl radical and/or an alkoxy radical, preferably a C₁-C₄-alkyl radical, more preferably a tert-butyl radical (t-Bu) and/or preferably a C₁-C₅-alkoxy radical, more preferably a methoxy radical.

It is advantageous when R¹ is not the same as R² and they are independently selected from C₁-C₁₀ alkyl, preferably C₁-C₅ alkyl, aryl, carboxamide and tosyl radicals.

In particularly preferred transition metal compounds of the formula Me(acac)(CO)L with Me=transition metal, L is selected from:

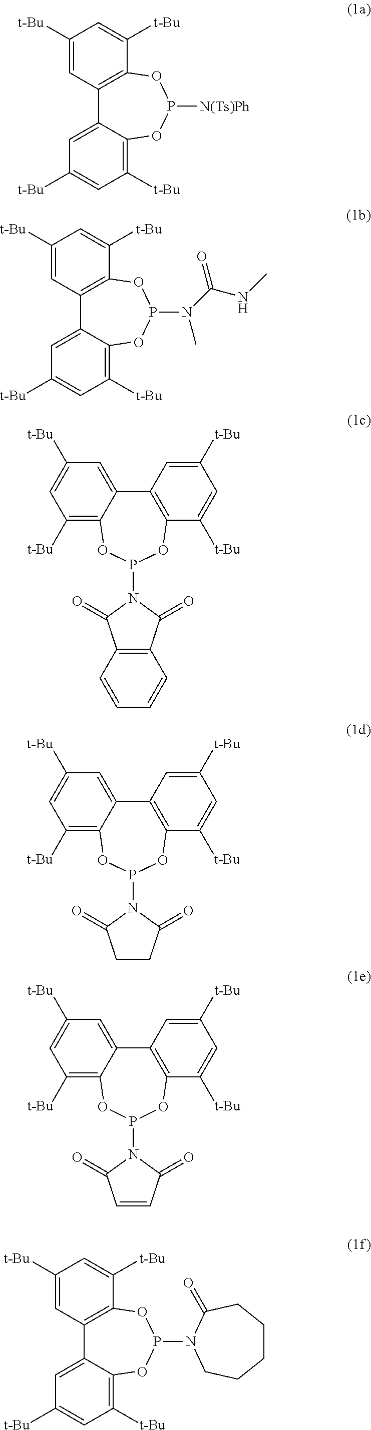

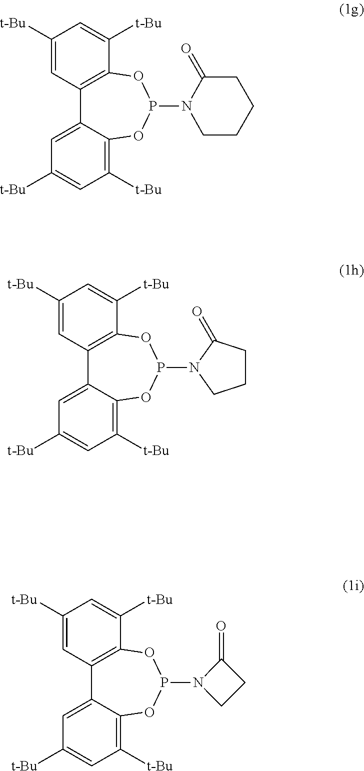 [SMILES: CC(C)(C)c1cc(C(C)(C)C)c2op(N3CCCCC3=O)oc3c(C(C)(C)C)cc(C(C)(C)C)cc3c2c1 ; CC(C)(C)c1cc(C(C)(C)C)c2op(N3CCCC3=O)oc3c(C(C)(C)C)cc(C(C)(C)C)cc3c2c1 ; CC(C)(C)c1cc(C(C)(C)C)c2op(N3CCC3=O)oc3c(C(C)(C)C)cc(C(C)(C)C)cc3c2c1]

Preferably, the transition metal Me is selected from ruthenium, cobalt, rhodium, iridium; especially preferably, Me=rhodium.

The transition metal is contacted with the inventive phosphoramidites as a precursor in the form of its salts, for example the halides, carboxylates—e.g. acetates—or commercially available complexes, for example acetylacetonates, carbonyls, cyclopolyenes—e.g. 1,5-cyclooctadiene—or else mixed forms thereof, for example Rh(acac)(CO)₂ with acac=acetylacetonate anion, Rh(acac)(COD) with COD=1,5-cyclooctadiene, and this reaction can be effected in a preceding reaction or else in the presence of a hydrogen- and carbon monoxide-containing gas mixture.

The present invention also provides catalytically active compositions in the hydroformylation comprising:

a) transition metal compounds of the formula Me(acac)(CO)L with Me=transition metal, where L is selected from:

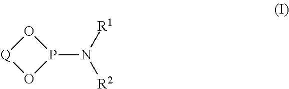

where Q is a divalent substituted or unsubstituted aromatic radical;
where R¹ is not the same as R² and they are independently selected from alkyl, aryl, carboxamide and organosulphonyl radicals;
or R¹ and R² together with N form a heterocyclic structure selected from lactams, dicarboximides;
preferably, Q is selected from substituted and unsubstituted 1,1′-biphenyl, 1,1′-binaphthyl and ortho-phenyl radicals;
more preferably, Q is selected from substituted and unsubstituted 1,1′-biphenyl radicals; preferred substituted 1,1′-biphenyl radicals are those having, in the 3,3′ and/or 5,5′ positions of the 1,1′-biphenyl-2,2′-diol base skeleton, an alkyl radical and/or an alkoxy radical, preferably a C₁-C₄-alkyl radical, more preferably a tert-butyl radical (t-Bu) and/or preferably a C₁-C₅-alkoxy radical, more preferably a methoxy radical;
at the same time, it is advantageous when R¹ is not the same as R² and they are independently selected from C₁-C₁₀-alkyl, preferably C₁-C₅-alkyl, aryl, carboxamide and tosyl radicals;
in particularly preferred transition metal compounds of the formula Me(acac)(CO)L with Me=transition metal, L is selected from:

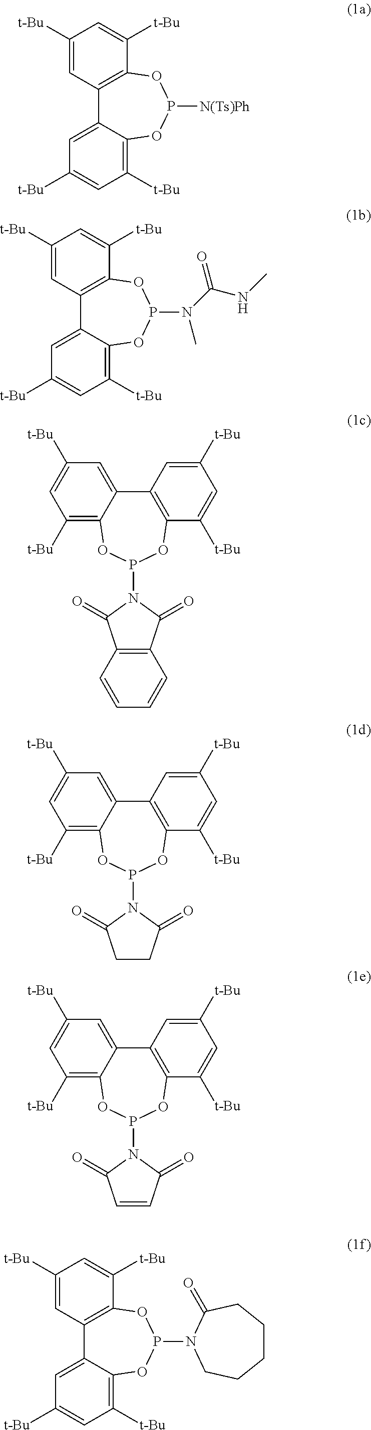

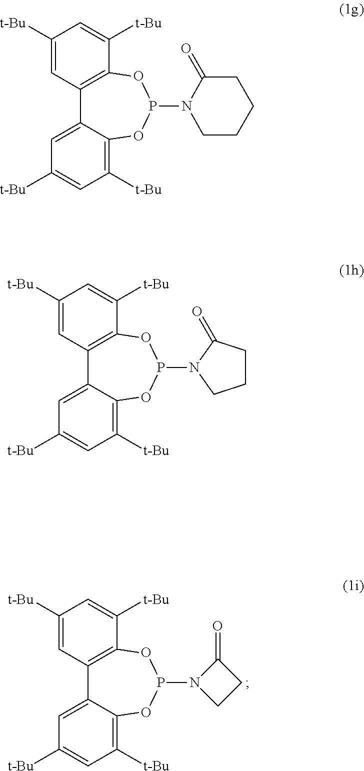 [SMILES: CC(C)(C)c1cc(C(C)(C)C)c2op(N3CCCCC3=O)oc3c(C(C)(C)C)cc(C(C)(C)C)cc3c2c1 ; CC(C)(C)c1cc(C(C)(C)C)c2op(N3CCCC3=O)oc3c(C(C)(C)C)cc(C(C)(C)C)cc3c2c1 ; CC(C)(C)c1cc(C(C)(C)C)c2op(N3CCC3=O)oc3c(C(C)(C)C)cc(C(C)(C)C)cc3c2c1]

preferably, the transition metal Me is selected from ruthenium, cobalt, rhodium, iridium; especially preferably, Me=rhodium;

b) free ligands of the formula (I):

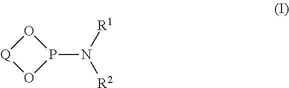

where Q is a divalent substituted or unsubstituted aromatic radical;
where R¹ is not the same as R² and the are independently selected from alkyl, aryl, carboxamide and organosulphonyl radicals;
or R¹ and R² together with N form a heterocyclic structure selected from lactams, dicarboximides;
preferably, Q is selected from substituted and unsubstituted 1,1′-biphenyl, 1,1′-binaphthyl and ortho-phenyl radicals;
more preferably, Q is selected from substituted and unsubstituted 1,1′-biphenyl radicals; preferred substituted 1,1′-biphenyl radicals are those having, in the 3,3′ and/or 5,5′ positions of the 1,1′-biphenyl-2,2′-diol base skeleton, an alkyl radical and/or an alkoxy radical, preferably a C₁-C₄-alkyl radical, more preferably a tert-butyl radical (t-Bu) and/or preferably a C₁-C₅-alkoxy radical, more preferably a methoxy radical;
at the same time, it is advantageous when R¹ is not the same as R² and they are independently selected from C₁-C₁₀-alkyl, preferably C₁-C₅-alkyl, aryl, carboxamide and tosyl radicals;
particularly preferred compounds of the formula (I) are selected from:

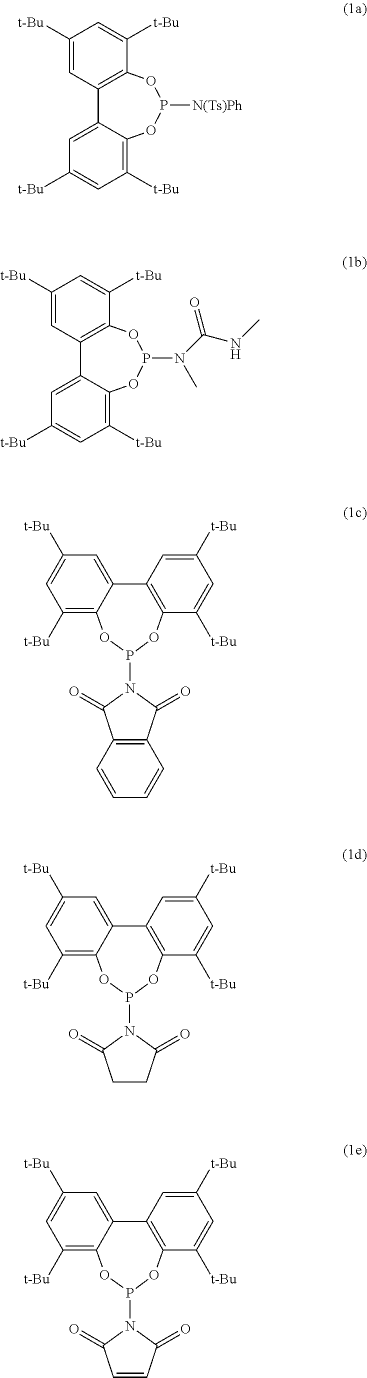

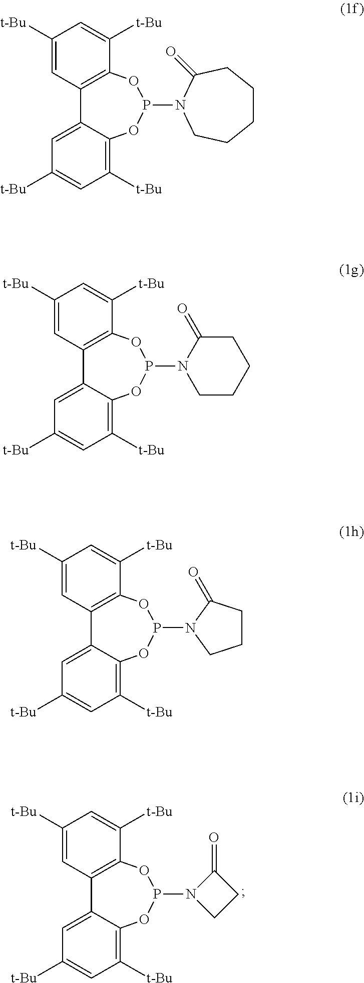 [SMILES: CC(C)(C)c1cc(C(C)(C)C)c2op(N3CCCCCC3=O)oc3c(C(C)(C)C)cc(C(C)(C)C)cc3c2c1 ; CC(C)(C)c1cc(C(C)(C)C)c2op(N3CCCCC3=O)oc3c(C(C)(C)C)cc(C(C)(C)C)cc3c2c1 ; CC(C)(C)c1cc(C(C)(C)C)c2op(N3CCCC3=O)oc3c(C(C)(C)C)cc(C(C)(C)C)cc3c2c1 ; CC(C)(C)c1cc(C(C)(C)C)c2op(N3CCC3=O)oc3c(C(C)(C)C)cc(C(C)(C)C)cc3c2c1]

c) solvents.

In the context of the present invention, solvents are regarded as being not only substances that have no inhibiting effect on product formation—having been added externally to the reaction mixture or initially charged therein—but also mixtures of compounds which form from side reactions or further reactions of the products in situ; for example what are called high boilers which form from the aldol condensation, the acetalization of the primary aldehyde product or else esterification, and lead to the corresponding aldol products, formates, acetals and ethers. Solvents initially charged externally in the reaction mixture may be aromatics, for example toluene-rich aromatics mixtures, or alkanes or mixtures of alkanes.

In general, high boilers are understood to mean those substances or else substance mixtures that boil at a higher temperature than the primary aldehyde product and have higher molar masses than the primary aldehyde product.

In the process according to the invention that has now been found, monodentate phosphoramidites which feature sulphonyl or lactam substituents or imides on the phosphorus are used for the first time as ligands in hydroformylation.

In the rhodium-catalysed hydroformylation of olefins, results achieved with the ligands prepared in accordance with the invention and under conditions selected in accordance with the invention are equally good or even better than with comparable monodentate phosphoramidite and phosphite ligands known from the literature.

The present invention further provides:

for the use of the catalytically active compositions in a process for hydroformylating unsaturated compounds and
a process for hydroformylating unsaturated compounds using said catalytically active composition, where the unsaturated compounds are preferably selected from:
hydrocarbon mixtures from steamcracking plants;
hydrocarbon mixtures from catalytically operated cracking plants;
hydrocarbon mixtures from oligomerization processes;
hydrocarbon mixtures comprising polyunsaturated compounds;
olefin-containing mixtures comprising olefins having up to 30 carbon atoms;
unsaturated carboxylic acid derivatives.

The unsaturated compounds which are hydroformylated in the process according to the invention include hydrocarbon mixtures obtained in petrochemical processing plants. Examples of these include what are called C₄ cuts. Typical compositions of C₄ cuts from which the majority of the polyunsaturated hydrocarbons has been removed and which can be used in the process according to the invention are listed in Table 1 below (see DE 10 2008 002188).

TABLE 1

| | Steamcracking plant | | Steamcracking plant | | Catalytic cracking plant | |
|---|---|---|---|---|---|---|
| Component | HCC₄ | HCC₄/SHP | Raff. I | Raff. I/SHP | CC₄ | CC₄/SHP |
| isobutane | 1-4.5 | 1-4.5 | 1.5-8 | 1.5-8 | 37 | 37 |
| [% by mass] | | | | | | |
| n-butane | 5-8 | 5-8 | 6-15 | 6-15 | 13 | 13 |
| [% by mass] | | | | | | |
| E-2-butene | 18-21 | 18-21 | 7-10 | 7-10 | 12 | 12 |
| [% by mass] | | | | | | |
| 1-butene | 35-45 | 35-45 | 15-35 | 15-35 | 12 | 12 |
| [% by mass] | | | | | | |
| isobutene | 22-28 | 22-28 | 33-50 | 33-50 | 15 | 15 |
| [% by mass] | | | | | | |
| Z-2-butene | 5-9 | 5-9 | 4-8 | 4-8 | 11 | 11 |
| [% by mass] | | | | | | |
| 1,3-butadiene | 500-8000 | 0-50 | 50-8000 | 0-50 | <10000 | 0-50 |
| [ppm by mass] | | | | | | |

Key:
HCC₄: typical of a C₄ mixture which is obtained from the C₄ cut from a steamcracking plant (high severity) after the hydrogenation of the 1,3-butadiene without additional moderation of the catalyst.
HCC₄/SHP: HCC₄ composition in which residues of 1,3-butadiene have been reduced further in a selective hydrogenation process/SHP.
Raff. I (raffinate I): typical of a C₄ mixture which is obtained from the C₄ cut from a steamcracking plant (high severity) after the removal of the 1,3-butadiene, for example by an NMP extractive rectification.
Raff. I/SHP: raff. I composition in which residues of 1,3-butadiene have been reduced further in a selective hydrogenation process/SHP.
CC₄: typical composition of a C₄ cut which is obtained from a catalytic cracking plant.
CC₄/SHP: composition of a C₄ cut in which residues of 1,3-butadiene have been reduced further in a selective hydrogenation process/SHP.

Likewise usable in the process according to the invention are unsaturated compounds or a mixture thereof selected from:
hydrocarbon mixtures from steamcracking plants;
hydrocarbon mixtures from catalytically operated cracking plants, for example FCC cracking plants;
hydrocarbon mixtures from oligomerization processes in the homogeneous phase and heterogeneous phases, for example the OCTOL, DIMERSOL, Fischer-Tropsch, Polygas, CatPoly, InAlk, Polynaphtha, Selectopol, MOGD, COD, EMOGAS, NExOCTANE or SHOP process;
hydrocarbon mixtures comprising polyunsaturated compounds;
unsaturated carboxylic acid derivatives.

Preferably, the unsaturated compounds or mixtures thereof used in the process according to the invention include unsaturated compounds having 2 to 30 carbon atoms, more preferably having 2 to 20 carbon atoms.

If polyunsaturated hydrocarbons or mixtures comprising them are used in the process according to the invention, the polyunsaturated hydrocarbons are preferably butadienes.

The unsaturated compounds which are hydroformylated in the process according to the invention additionally include unsaturated carboxylic acid derivatives. Preferably, these unsaturated carboxylic acid derivatives are selected from fatty acid esters; methyl oleate is particularly preferred.

Preferably, these fatty acid esters are based on renewable raw materials. In the context of the present invention, renewable raw materials, as opposed to petrochemical raw materials based on fossil resources, for example mineral oil or hard coal, are understood to mean those raw materials which arise or are produced on the basis of biomass. The terms “biomass”, “bio-based” or “based on”, or “produced from renewable raw materials”, include all materials of biological origin which originate from what is called the “short-term carbon cycle”, and are thus not part of geological formations or fossil strata. More particularly, “based on renewable raw materials” and “on the basis of renewable raw materials” are understood to mean that, by the ASTM D6866-08 method (¹⁴C method), the appropriate proportion of ¹⁴C isotopes can be detected in the hydroformylation mixture of the fatty acid esters.

The identification and quantification of renewable raw materials can be effected to ASTM Method D6866. One characterizing feature of renewable raw materials is the proportion therein of the ¹⁴C carbon isotope as against petrochemical raw materials. With the aid of the radiocarbon method, it is possible to determine the proportion of ¹⁴C isotopes and hence also the proportion of molecules based on renewable raw materials.

If olefins or olefin-containing mixtures are used as unsaturated hydrocarbons in the process according to the invention, the olefins are preferably selected from n-octenes, 1-octene and C₈-containing olefin mixtures.

In the process according to the invention, preferably, in a first process step, phosphoramidites of the formula (I):

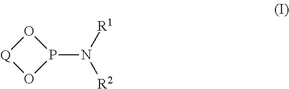

where Q is a divalent substituted or unsubstituted aromatic radical;
where R¹ is not the same as R² and they are independently selected from alkyl, aryl, carboxamide and organosulphonyl radicals;
or R¹ and R² together with N form a heterocyclic structure selected from lactams, dicarboximides;
preferably, Q is selected from substituted or unsubstituted 1,1′-biphenyl, 1,1′-binaphthyl and ortho-phenyl radicals; more preferably, Q is selected from substituted or unsubstituted 1,1′-biphenyl radicals;
preferred substituted 1,1′-biphenyl radicals are those having, in the 3,3′ and/or 5,5′ positions of the 1,1′-biphenyl-2,2′-diol base skeleton, an alkyl radical and/or an alkoxy radical, preferably a C₁-C₄-alkyl radical, more preferably a tert-butyl radical (t-Bu) and/or preferably a C₁-C₅-alkoxy radical, more preferably a methoxy radical; at the same time, it is advantageous when R¹ is not the same as R² and they are independently selected from C₁-C₁₀-alkyl, preferably C₁-C₅-alkyl, aryl, carboxamide and tosyl radicals;
particularly preferred compounds of the formula (I) are selected from:

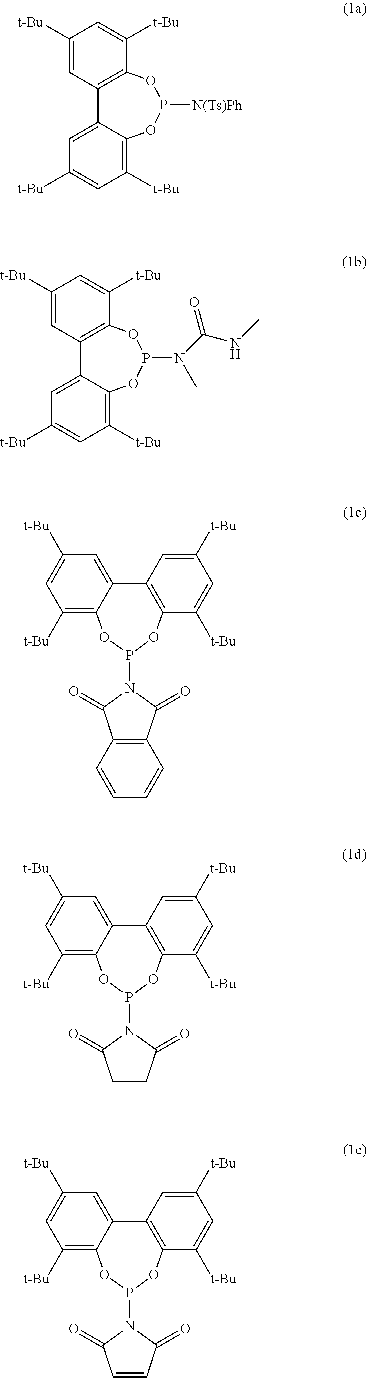

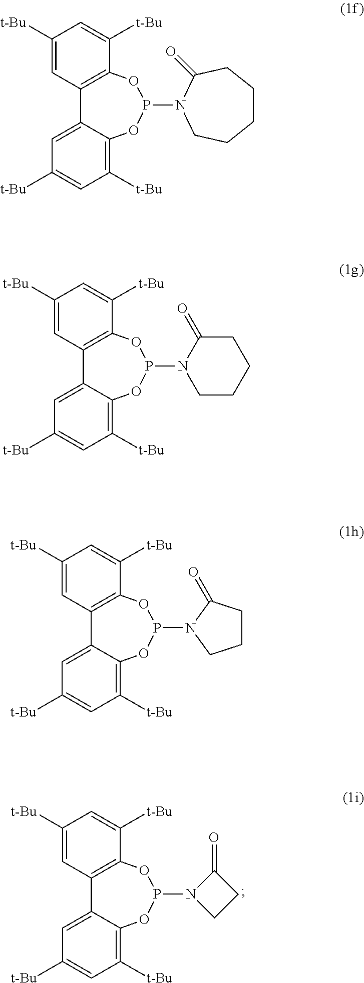 [SMILES: CC(C)(C)c1cc(C(C)(C)C)c2op(N3CCCCCC3=O)oc3c(C(C)(C)C)cc(C(C)(C)C)cc3c2c1 ; CC(C)(C)c1cc(C(C)(C)C)c2op(N3CCCCC3=O)oc3c(C(C)(C)C)cc(C(C)(C)C)cc3c2c1 ; CC(C)(C)c1cc(C(C)(C)C)c2op(N3CCCC3=O)oc3c(C(C)(C)C)cc(C(C)(C)C)cc3c2c1 ; CC(C)(C)c1cc(C(C)(C)C)c2op(N3CCC3=O)oc3c(C(C)(C)C)cc(C(C)(C)C)cc3c2c1]

are initially charged as ligands in at least one reaction zone, reacted with a precursor of the transition metal to give a transition metal compound of the formula Me(acac)(CO)L where L is selected from:

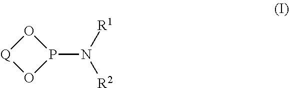

where Q is a divalent substituted or unsubstituted aromatic radical,
where R¹ is not the same as R² and they are independently selected from alkyl, aryl, carboxamide and organosulphonyl radicals;
or R¹ and R² together with N form a heterocyclic structure selected from lactans, dicarboximides;
preferably, Q is selected from substituted or unsubstituted 1,1′-biphenyl, 1,1′-binaphthyl and ortho-phenyl radicals; more preferably, Q is selected from substituted or unsubstituted 1,1′-biphenyl radicals;
preferred substituted 1,1′-biphenyl radicals are those having, in the 3,3′ and/or 5,5′ positions of the 1,1′-biphenyl-2,2′-diol base skeleton, an alkyl radical and/or an alkoxy radical, preferably a C₁-C₄-alkyl radical, more preferably a tert-butyl radical (t-Bu) and/or preferably a C₁-C₅-alkoxy radical, more preferably a methoxy radical; at the same time, it is advantageous when R¹ is not the same as R² and they are independently selected from C₁-C₁₀-alkyl, preferably C₁-C₅-alkyl, aryl, carboxamide and tosyl radicals;
in particularly preferred transition metal compounds of the formula Me(acac)(CO)L with Me=transition metal, L is selected from:

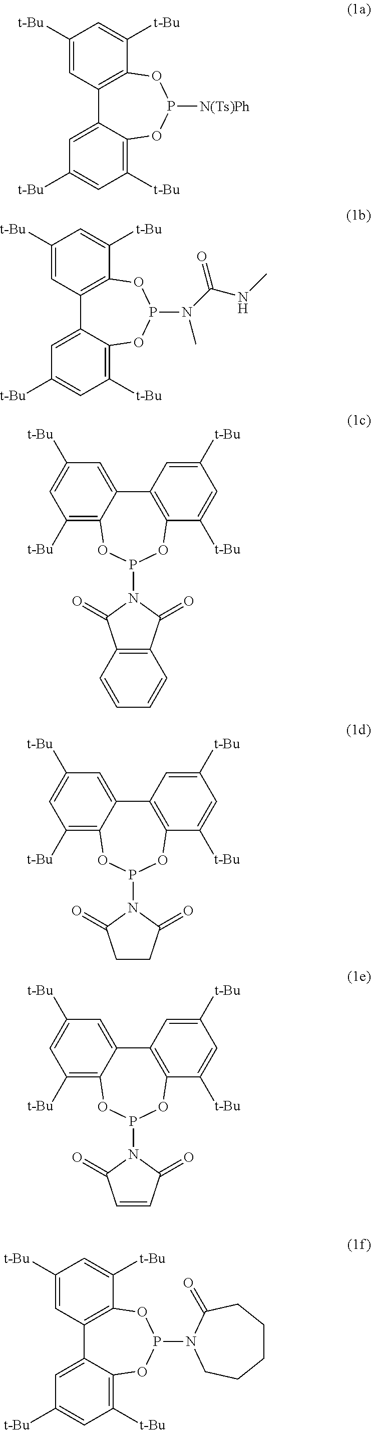

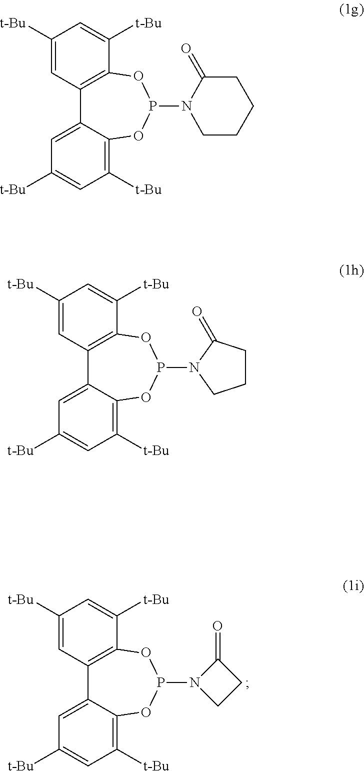 [SMILES: CC(C)(C)c1cc(C(C)(C)C)c2op(N3CCCCC3=O)oc3c(C(C)(C)C)cc(C(C)(C)C)cc3c2c1 ; CC(C)(C)c1cc(C(C)(C)C)c2op(N3CCCC3=O)oc3c(C(C)(C)C)cc(C(C)(C)C)cc3c2c1 ; CC(C)(C)c1cc(C(C)(C)C)c2op(N3CCC3=O)oc3c(C(C)(C)C)cc(C(C)(C)C)cc3c2c1]

preferably, the transition metal Me is selected from ruthenium, cobalt, rhodium, iridium; especially preferably, Me=rhodium;
and optional, preferably compulsory, further addition of free ligands of the formula (I):

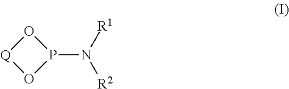

where Q is a divalent substituted or unsubstituted aromatic radical,
where R¹ is not the same as R² and they are independently selected from alkyl, aryl, carboxamide and organosulphonyl radicals;
or R¹ and R² together with N form a heterocyclic structure selected from lactams, dicarboximides;
preferably, Q is selected from substituted or unsubstituted 1,1′-biphenyl, 1,1′-binaphthyl and ortho-phenyl radicals; more preferably, Q is selected from substituted or unsubstituted 1,1′-biphenyl radicals;
preferred substituted 1,1′-biphenyl radicals are those having, in the 3,3′ and/or 5,5′ positions of the 1,1′-biphenyl-2,2′-diol base skeleton, an alkyl radical and/or an alkoxy radical, preferably a C₁-C₄-alkyl radical, more preferably a tert-butyl radical (t-Bu) and/or preferably a C₁-C₅-alkoxy radical, more preferably a methoxy radical;
at the same time, it is advantageous when R¹ is not the same as R² and they are independently selected from C₁-C₁₀-alkyl, preferably C₁-C₅-alkyl, aryl, carboxamide and tosyl radicals;
particularly preferred compounds of the formula (I) are selected from:

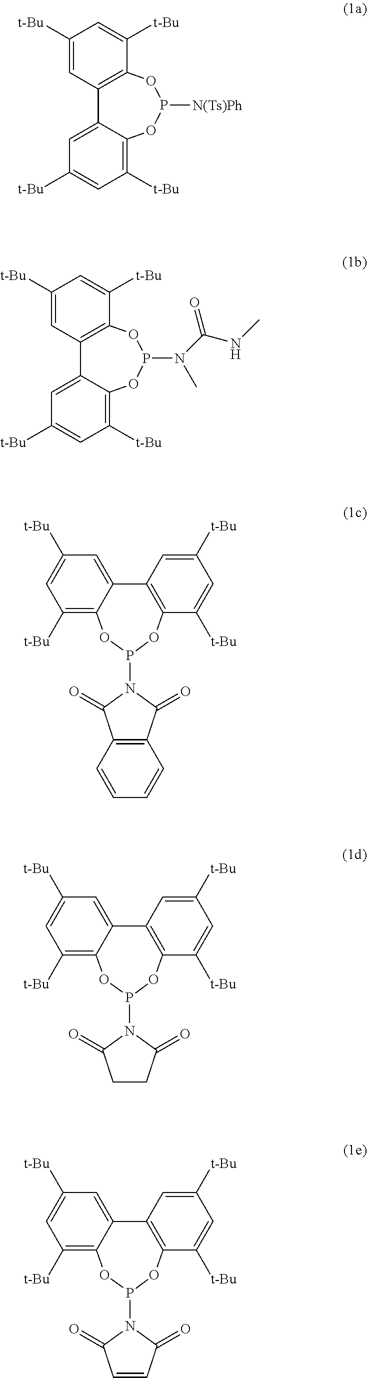

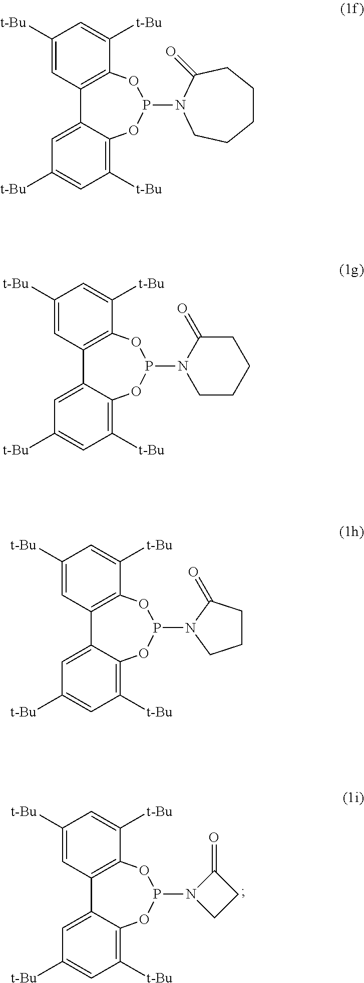 [SMILES: CC(C)(C)c1cc(C(C)(C)C)c2op(N3CCCCCC3=O)oc3c(C(C)(C)C)cc(C(C)(C)C)cc3c2c1 ; CC(C)(C)c1cc(C(C)(C)C)c2op(N3CCCCC3=O)oc3c(C(C)(C)C)cc(C(C)(C)C)cc3c2c1 ; CC(C)(C)c1cc(C(C)(C)C)c2op(N3CCCC3=O)oc3c(C(C)(C)C)cc(C(C)(C)C)cc3c2c1 ; CC(C)(C)c1cc(C(C)(C)C)c2op(N3CCC3=O)oc3c(C(C)(C)C)cc(C(C)(C)C)cc3c2c1]

and also solvents and a carbon monoxide- and hydrogen-containing gas mixture, to give a catalytically active composition in the hydroformylation;
in a subsequent step, the unsaturated compounds are added under the reaction conditions to form a polyphasic reaction mixture;
after the end of the reaction, the reaction mixture is separated into aldehydes, alcohols, high boilers, ligands and/or, preferably and, degradation products of the catalytically active composition.

In the process according to the invention, the unsaturated compound(s) are preferably added together with the precursor of the transition metal and the ligands (compounds of the formula (I); this is especially preferred when the unsaturated compound(s) are in a liquid state of matter at room temperature and standard pressure corresponding to 1013 hPa.

The hydroformylation is conducted under customary reaction conditions, preference being given to a temperature of 60° C. to 160° C. and a syngas pressure of 1.0 MPa to MPa; particular preference is given to a temperature of 60° C. to 120° C. and a syngas pressure of 1.0 MPa to 6.0 MPa.

In the context of this invention, degradation products are regarded as being substances which originate from the breakdown of the composition catalytically active in the hydroformylation. They are described, for example, in U.S. Pat. No. 5,364,950, U.S. Pat. No. 5,763,677, and also in Catalyst Separation, Recovery and Recycling”, edited by D. J. Cole-Hamilton, R. P. Tooze, 2006, NL, pages 25-26, and in Rhodium-catalyzed Hydroformylation, ed. by P. W. N. M. van Leeuwen and C. Claver, Kluwer Academic Publishers 2006, AA Dordrecht, NL, pages 206-211.

The present invention finally provides a polyphasic reaction mixture comprising:
unsaturated compounds;
a gas mixture including carbon monoxide, hydrogen;
catalytically active compositions comprising:

a) transition metal compounds of the formula Me(acac)(CO)L with Me=transition metal, where L is selected from:

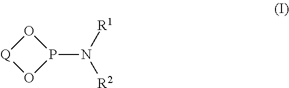

where Q is a divalent substituted or unsubstituted aromatic radical;
where R¹ is not the same as R² and they are independently selected from alkyl, aryl, carboxamide and organosulphonyl radicals;
or R¹ and R² together with N form a heterocyclic structure selected from lactams, dicarboximides;
preferably, Q is selected from substituted and unsubstituted 1,1′-biphenyl, 1,1′-binaphthyl and ortho-phenyl radicals;
more preferably, Q is selected from substituted and unsubstituted 1,1′-biphenyl radicals; preferred substituted 1,1′-biphenyl radicals are those having, in the 3,3′ and/or 5,5′ positions of the 1,1′-biphenyl-2,2′-diol base skeleton, an alkyl radical and/or an alkoxy radical, preferably a C₁-C₄-alkyl radical, more preferably a tert-butyl radical (t-Bu) and/or preferably a C₁-C₅-alkoxy radical, more preferably a methoxy radical; at the same time, it is advantageous when R¹ is not the same as R² and they are independently selected from C₁-C₁₀-alkyl, preferably C₁-C₅-alkyl, aryl, carboxamide and tosyl radicals;
in particularly preferred transition metal compounds of the formula Me(acac)(CO)L with Me=transition metal, L is selected from:

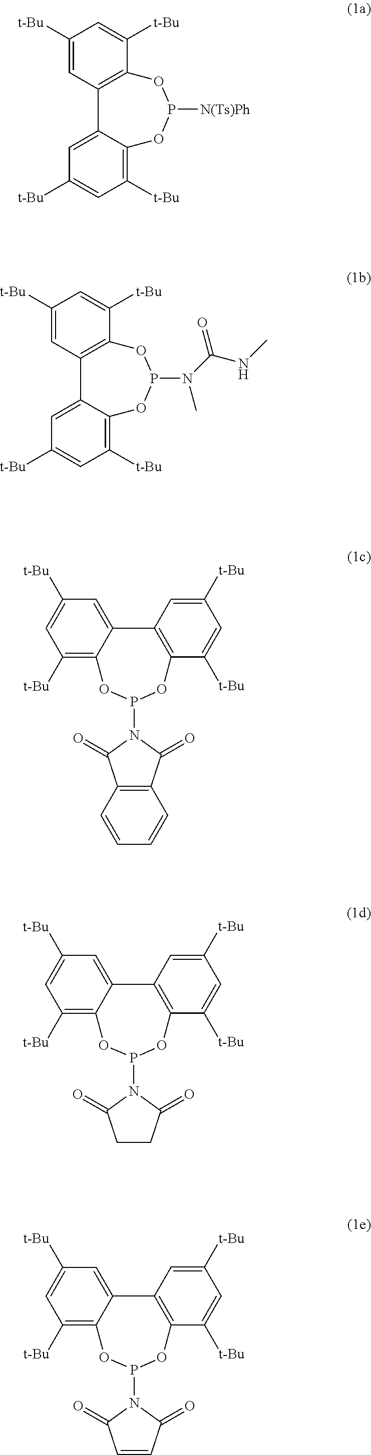

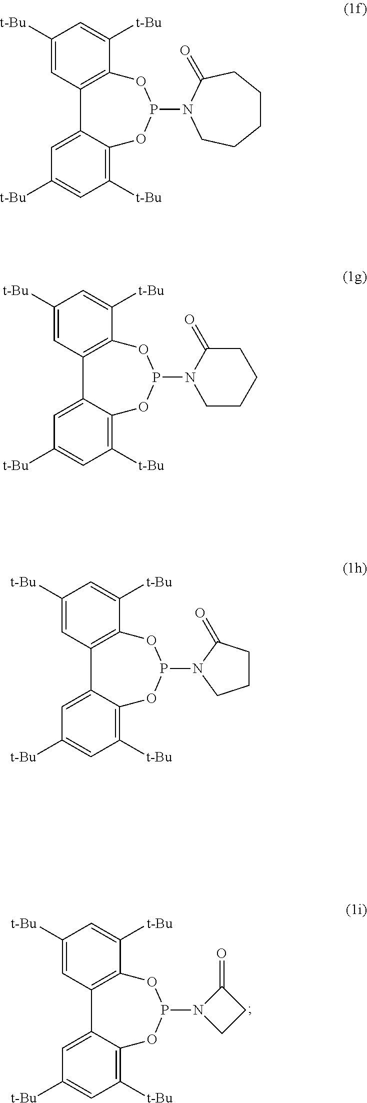 [SMILES: CC(C)(C)c1cc(C(C)(C)C)c2op(N3CCCCCC3=O)oc3c(C(C)(C)C)cc(C(C)(C)C)cc3c2c1 ; CC(C)(C)c1cc(C(C)(C)C)c2op(N3CCCCC3=O)oc3c(C(C)(C)C)cc(C(C)(C)C)cc3c2c1 ; CC(C)(C)c1cc(C(C)(C)C)c2op(N3CCCC3=O)oc3c(C(C)(C)C)cc(C(C)(C)C)cc3c2c1 ; CC(C)(C)c1cc(C(C)(C)C)c2op(N3CCC3=O)oc3c(C(C)(C)C)cc(C(C)(C)C)cc3c2c1]

preferably, the transition metal Me is selected from ruthenium, cobalt, rhodium, iridium; especially preferably, Me=rhodium;

b) free ligands of the formula (I):

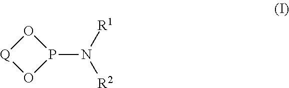

where Q is a divalent substituted or unsubstituted aromatic radical;
where R¹ is not the same as R² and they are independently selected from alkyl, aryl, carboxamide and organosulphonyl radicals;
or R¹ and R² together with N form a heterocyclic structure selected from lactams, dicarboximides;
preferably, Q is selected from substituted and unsubstituted 1,1′-biphenyl, 1,1′-binaphthyl and ortho-phenyl radicals;
more preferably, Q is selected from substituted and unsubstituted 1,1′-biphenyl radicals; preferred substituted 1,1′-biphenyl radicals are those having, in the 3,3′ and/or 5,5′ positions of the 1,1′-biphenyl-2,2′-diol base skeleton, an alkyl radical and/or an alkoxy radical, preferably a C₁-C₄-alkyl radical, more preferably a tert-butyl radical (t-Bu) and/or preferably a C₁-C₅-alkoxy radical, more preferably a methoxy radical;
at the same time, it is advantageous when R¹ is not the same as R² and they are independently selected from C₁-C₁₀-alkyl, preferably C₁-C₅-alkyl, aryl, carboxamide and tosyl radicals;
particularly preferred compounds of the formula (I) are selected from:

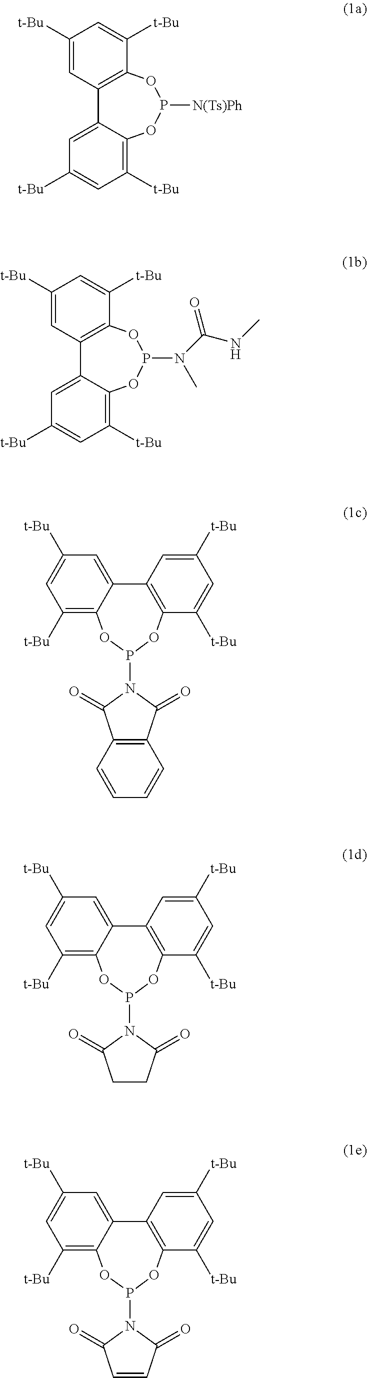

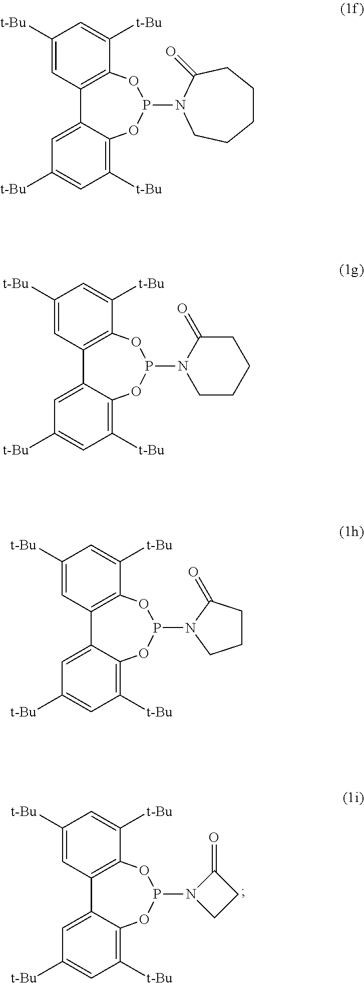 [SMILES: CC(C)(C)c1cc(C(C)(C)C)c2op(N3CCCCCC3=O)oc3c(C(C)(C)C)cc(C(C)(C)C)cc3c2c1 ; CC(C)(C)c1cc(C(C)(C)C)c2op(N3CCCCC3=O)oc3c(C(C)(C)C)cc(C(C)(C)C)cc3c2c1 ; CC(C)(C)c1cc(C(C)(C)C)c2op(N3CCCC3=O)oc3c(C(C)(C)C)cc(C(C)(C)C)cc3c2c1 ; CC(C)(C)c1cc(C(C)(C)C)c2op(N3CCC3=O)oc3c(C(C)(C)C)cc(C(C)(C)C)cc3c2c1]

c) solvents;

where the unsaturated compounds are selected from:
hydrocarbon mixtures from steamcracking plants;
hydrocarbon mixtures from catalytically operated cracking plants;
for example FCC cracking plants;
hydrocarbon mixtures from oligomerization processes in the homogeneous phase and heterogeneous phases, for example the OCTOL, DIMERSOL, Fischer-Tropsch, Polygas, CatPoly, InAlk, Polynaphtha, Selectopol, MOGD, COD, EMOGAS, NExOCTANE or SHOP process;
hydrocarbon mixtures comprising polyunsaturated compounds;
unsaturated carboxylic acid derivatives;
where the solvent is added externally and does not intervene in an inhibiting fashion in the hydroformylation reaction, especially when the solvent is formed in situ from the primary products.

### EXAMPLES

### General Working Methods

All the preparations which follow were conducted with standard Schlenk technology under protective gas. The solvents were dried over suitable desiccants before use (Purification of Laboratory Chemicals, W. L. F. Armarego (Author), Christina Chai (Author), Butterworth Heinemann (Elsevier), 6th edition, Oxford 2009). Phosphorus trichloride (Aldrich) was distilled under argon before use. All preparative operations were effected in baked-out vessels. The products were characterized by means of NMR spectroscopy. Chemical shifts are reported in ppm. The ³¹P NMR signals were referenced according to: SR_{31P}=SR_{1H}*(BF_{31P}/BF_{1H})=SR_{1H}*0.4048. (Robin K. Harris, Edwin D. Becker, Sonia M. Cabral de Menezes, Robin Goodfellow, and Pierre Granger, Pure Appl. Chem., 2001, 73, 1795-1818; Robin K. Harris, Edwin D. Becker, Sonia M. Cabral de Menezes, Pierre Granger, Roy E. Hoffman and Kurt W. Zilm, Pure Appl. Chem., 2008, 80, 59-84).

The recording of nuclear resonance spectra was effected on Bruker Avance 300 or Bruker Avance 400, gas chromatography analysis on Agilent GC 7890A, elemental analysis on Leco TruSpec CHNS and Varian ICP-OES 715, and ESI-TOF mass spectrometry on Thermo Electron Finnigan MAT 95-XP and Agilent 6890 N/5973 instruments.

### Example 1

### General Synthesis Method

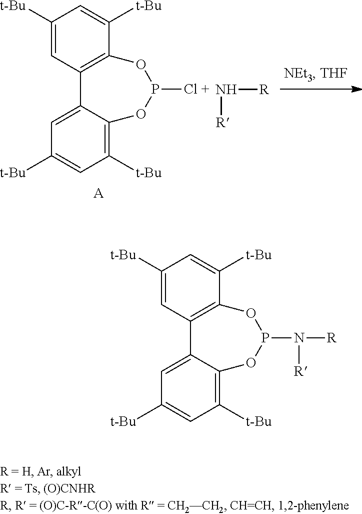

To a stirred solution of the chlorophosphite A (2 mmol) (preparation according to US20080188686 A1) in dried THF (10 ml) were added Et₃N (3 mmol) and the appropriate lactam, sulphonamide, dicarboximide or urea derivative (2.3 mmol) in dried THF (10 ml). The solution was stirred at room temperature. The progress of the reaction was monitored by means of ³¹P NMR spectroscopy. Once the chlorophosphite had been fully converted (4-24 h), the readily evaporable liquids were distilled off under reduced pressure. Subsequently, dried toluene (10 ml) was added. The resultant suspension was filtered through a layer of neutral alumina (about 2 cm, Ø=2 cm; Schlenk filter, porosity 4) and then washed through with toluene (2×7 ml). After the solution had been concentrated, the residue was dried under reduced pressure at 45-50° C. for 3 h. The products were pure enough to be usable for catalysis and hydrolysis tests without further purifying operations.

### Example 2

### N-(2,4,8,10-Tetra-tert-butyldibenz[d,f]{1,3,2}dioxaphosphepin-6-yl)-N-tosylaniline

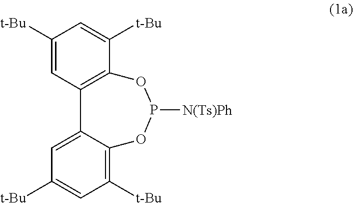

Yield: 63%; white solid. ¹H NMR (300 MHz, CDCl₃): δ 1.20 (s, 18H), 1.45 (s, 18H), 2.36 (s, 3H), 6.25 (d, 2H, J=7.7 Hz), 6.72 (t, 2H, J=7.7 Hz), 6.78 (d, 2H, J=2.3 Hz), 6.80-6.87 (m, 1H), 7.19 (d, 2H, J=8.3 Hz), 7.34 (d, 2H, J=2.4 Hz), 7.59 (d, 2H, J=8.3 Hz). ³¹P NMR (121 MHz, CDCl₃): δ 129.9 (s). ¹³C NMR (62 MHz, CDCl₃): δ 21.7 (s, CH₃PhSO₂), 31.4-31.5 (overlapping singlet and doublet, J=2.8 Hz, 2 types of (CH₃)₃C), 34.6 (s, (CH₃)₃C), 35.5 (s, (CH₃)₃C), 124.3 (s, CH_{Ar}), 126.5 (s, CH_{Ar}), 127.4 (s, CH_{Ar}), 127.9 (2 overlapping singlets, 2×CH_{Ar}), 129.6 (s, CH_{Ar}), 130.8 (s, CH_{Ar}), 132.3 (d, J=3.8 Hz, C_{Ar}), 135.5 (d, J=4.8 Hz, C_{Ar}), 137.6 (s, C_{Ar}), 139.9 (d, J=1.9 Hz, C_{Ar}), 143.9 (s, C_{Ar}), 145.9 (d, J=5.9 Hz, C_{Ar}), 146.8 (s, C_{Ar}). HRMS (EI): calculated m/z (C₄₁H₅₂N₁O₄P₁S₁) 685.334989; found 685.33492; HRMS (ESI-TOF/MS): calculated m/z (C₄₁H₅₃N₁O₄P₁S₁, (M+H)⁺) 686.34274; found 686.34391; calculated m/z (C₄₁H₅₂N₁Na₁O₄P₁S₁, (M+Na)⁺) 708.32469; found 708.32644. MS (EI, 70 eV): m/z (I, %): 685 (35), 621 (75), 546 (51), 439 (100), 246 (20), 91 (43), 57 (83).

### Example 3

### N-(2,4,8,10-Tetra-tert-butyldibenz[d,f]{1,3,2}dioxaphosphepin-6-yl)phthalimide

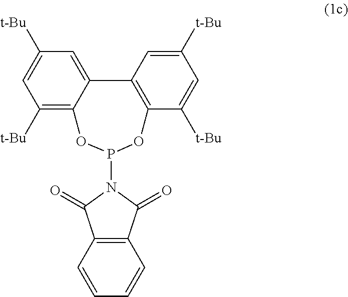 [SMILES: CC(C)(C)c1cc(C(C)(C)C)c2op(N3C(=O)c4ccccc4C3=O)oc3c(C(C)(C)C)cc(C(C)(C)C)cc3c2c1]

Yield: 96%; white solid. ¹H NMR (300 MHz, CDCl₃): δ 1.29-1.30 (2 overlapping singlets, 36H), 7.18 (d, 2H, J=2.5 Hz), 7.32 (d, 2H, J=2.5 Hz), 7.64-7.69 (m, 2H), 7.72-7.80 (m, 2H). ³¹P NMR (121 MHz, CDCl₃): δ 131.1 (s). ¹³C NMR (75 MHz, CDCl₃): δ 31.0 (d, J=2.4 Hz, (CH₃)₃C), 31.6 (s, CH₃)₃C), 34.7 (s, (CH₃)C₃), 35.4 (s, (CH₃)₃C), 124.0 (s, CH_{Ar}), 124.3 (s, CH_{Ar}), 127.0 (s, CH_{Ar}), 132.7-132.8 (2 overlapping singlets, 2 types of C_{Ar}), 134.6 (s, CH_{Ar}), 139.5 (s, C_{Ar}), 146.7 (s, C_{Ar}), 147.5 (d, J=5.9 Hz, C_{Ar}), 168.7 (S, C═O). HRMS (EI): calculated m/z (C₃H₄₄N₁O₄P₁) 585.30025; found 585.299809; MS (EI, 70 eV): m/z (I, %): 585 (77), 570 (58), 528 (11), 441 (13), 423 (41), 57 (100).

### Example 4

### N-(2,4,8,10-Tetra-tert-butyldibenz[d,f]{1,3,2}dioxaphosphepin-6-yl)succinimide

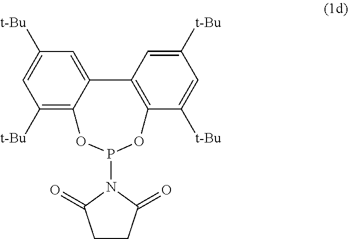 [SMILES: CC(C)(C)c1cc(C(C)(C)C)c2op(N3C(=O)CCC3=O)oc3c(C(C)(C)C)cc(C(C)(C)C)cc3c2c1]

Yield: 95%; white solid. ¹H NMR (300 MHz, CDCl₃): δ 1.28 (s, 18H), 1.34 (s, 18H), 2.58 (s, 4H), 7.14 (d, 2H, J=2.3 Hz), 7.32 (d, 2H, J=2.3 Hz). ³¹P NMR (121 MHz, CDCl₃): δ 131.7 (s). ¹³C NMR (62 MHz, CDCl₃): δ 29.6 (d, ³J=2.9 Hz, CH₂), 30.9 (d, J=2.5 Hz, (CH_)₃C), 31.5 (s, (CH₃)₃C), 34.7 (s, (CH₃)₃C), 35.4 (s, (CH₃)₃C), 124.3 (s, CH_{Ar}), 127.0 (s, CH_{Ar}), 132.5 (d, J=4.4 Hz, C_{Ar}), 139.2 (d, J=2.3 Hz, C_{Ar}), 147.0 (S, C_{Ar}), 147.3 (d, J=5.8 Hz, C_{Ar}), 178.0 (s, C═O). HRMS (ESI): calculated m/z (C₃₂H₄₅N₁O₄P₁, (M+H)⁺) 538.30807; found 538.30813; MS (EI, 70 eV): m/z (I, %): 537 (100), 522 (39), 480 (20), 423 (84), 57 (35).

### Example 5

### N-(2,4,8,10-Tetra-tert-butyldibenz[d,f]{1,3,2}dioxaphosphepin-6-yl)maleimide

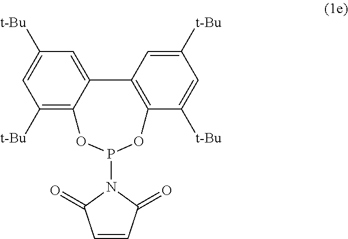 [SMILES: CC(C)(C)c1cc(C(C)(C)C)c2op(N3C(=O)C=CC3=O)oc3c(C(C)(C)C)cc(C(C)(C)C)cc3c2c1]

Yield: 96%; white solid. ¹H NMR (300 MHz, CDCl₃): δ 1.28 (s, 18H), 1.32 (s, 18H), 6.60 (s, 2H), 7.15 (d, 2H, J=2.4 Hz), 7.32 (d, 2H, J=2.5 Hz). ³¹P NMR (121 MHz, CDCl₃): δ 131.26 (s). ¹³C NMR (75 MHz, CDCl₃): δ 30.9 (d, J=2.6 Hz, (CH₃)₃C), 31.5 (s, (CH₃)₃C), 34.7 (s, (CH₃)₃C), 35.4 (s, (CH₃)₃C), 124.7 (s, CH_{Ar}), 127.0 (s, CH_{Ar}), 132.7 (d, J=3.8 Hz, C_{Ar}), 136.0 (d, J=2.5 Hz, C═C), 139.4 (d, J=2.1 Hz, C_{Ar}), 147.0 (s, C_{Ar}), 147.1 (s, C_{Ar}), 171.4 (s, C═O). HRMS (ESI-TOF/MS): calculated m/z (C₃₂H₄₃NO₄P, (M+H)⁺) 536.29242; found 536.29178; calculated m/z (C₃₂H₄₂N₁Na₁O₄P₁, (M+Na)⁺) 558.27437; found 558.27382. MS (EI, 70 eV): m/z (I, %): 535 (100), 520 (51), 441 (11), 423 (29), 57 (40).

### Example 6

### N-(2,4,8,10-Tetra-tert-butyldibenz[d,f]{1,3,2}dioxaphosphepin-6-yl)-ε-caprolactam

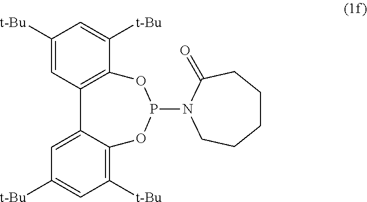 [SMILES: CC(C)(C)c1cc(C(C)(C)C)c2op(N3CCCCCC3=O)oc3c(C(C)(C)C)cc(C(C)(C)C)cc3c2c1]

Yield: 96%; white solid. ¹H NMR (250 MHz, CDCl₃): δ 1.27 (s, 18H), 1.37 (s, 18H of t-Bu+2H of CH₂), 1.49-1.59 (m, 2H), 1.61-1.73 (m, 2H), 2.43-2.51 (m, 2H), 2.96-3.04 (m, 2H), 7.09 (d, 2H, J=2.4 Hz), 7.36 (d, 2H, J=2.4 Hz). ³¹P NMR (101 MHz, CDCl₃): δ 132.87 (s). ¹³C NMR (62 MHz, CDCl₃): δ 23.4 (s, CH₂), 29.7 (s, CH₂), 29.8 (s, CH₂), 31.1 (d, J=2.8 Hz, (CH₃)₃C), 31.5 (s, (CH₃)₃C), 34.7 (s, (CH₃)₃C), 35.5 (s, (CH₃)₃C), 38.8 (s, CH₂), 43.9 (d, J=5.2 Hz, CH₂), 124.5 (s, CH_{Ar}), 126.6 (s, CH_{Ar}), 132.6 (d, J=3.7 Hz, C_{Ar}), 140.2 (d, J=1.6 Hz, C_{Ar}), 146.6 (s, C_{Ar}), 146.7 (s, C_{Ar}), 182.7 (d, ²J=18.4 Hz, C═O). HRMS (EI): calculated m/z (C₃₄H₅₀N₁O₃P₁) 551.35228; found 551.35208; MS (EI, 70 eV): m/z (I, %): 551 (9), 536 (26), 494 (77), 441 (31), 91 (100), 57 (26).

### Example 7

### N-(2,4,8,10-Tetra-tert-butyldibenz[d,f]{1,3,2}dioxaphosphepin-6-yl)-δ-valerolactam

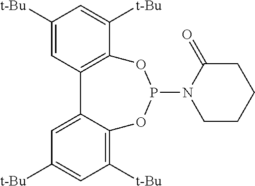 [SMILES: CC(C)(C)c1cc(C(C)(C)C)c2op(N3CCCCC3=O)oc3c(C(C)(C)C)cc(C(C)(C)C)cc3c2c1]

Yield: 90%; white solid. ¹H NMR (300 MHz, CDCl₃): δ 1.29 (s, 18H), 1.38 (s, 18H), 1.39-1.41 (m, 2H), 1.58-1.71 (m, 2H), 2.38 (t, 2H, J=6.8 Hz), 2.92-3.01 (m, 2H), 7.09 (d, 2H, J=2.4 Hz), 7.36 (d, 2H, J=2.4 Hz). ³¹P NMR (121 MHz, CDCl₃): δ 132.6 (s). ¹³C NMR (75 MHz, CDCl₃): δ 19.8 (s, CH₂), 22.6 (s, CH₂), 31.1 (d, J=2.7 Hz, (CH₃)₃C), 31.5 (s, (CH₃)₃C), 34.7 (s, (CH₃)₃C), 35.5 (s, (CH₃)₃C), 33.3 (d, J=2.2 Hz, CH₂), 42.9 (d, J=4.9 Hz, CH₂), 124.4 (s, CH_{Ar}), 126.6 (s, CH_{Ar}), 132.6 (d, J=4.0 Hz, C_{Ar}), 140.1 (d, J=1.6 Hz, C_{Ar}), 146.7 (s, C_{Ar}), 146.9 (d, J=5.2 Hz, C_{Ar}), 177.4 (d, ²J=17.5 Hz, C═O). MS (EI, 70 eV): m/z (I, %): 537 (4), 522 (19), 480 (100), 140 (76), 57 (20). HRMS (EI): calculated m/z (C₃₃H₈N₁O₃P₁) 537.33663; found 537.33652. Anal. calculated for C₃₃H₄N₃O₁P₁: C, 73.71; H, 9.00; N, 2.60; P, 5.76. Found: C, 73.74; H, 8.77; N, 2.55; P, 5.45.

### Example 8

### N-(2,4,8,10-Tetra-tert-butyldibenz[d,f]{1,3,2}dioxaphosphepin-6-yl)-γ-butyrolactam

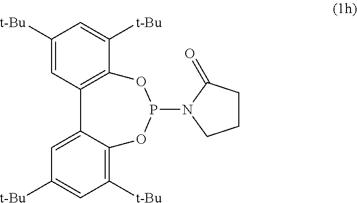 [SMILES: CC(C)(C)c1cc(C(C)(C)C)c2op(N3CCCC3=O)oc3c(C(C)(C)C)cc(C(C)(C)C)cc3c2c1]

Yield: 90%; white solid. ¹H NMR (300 MHz, CD₂Cl₂): δ 1.42 (s, 18H); 1.51 (s, 18H); 1.91 (m, 2H); 2.41 (m, 2H); 3.14 (m, 2H); 7.24 (d, 2H, ⁴J_{HH}=2.4 Hz); 7.52 (d, 2H, ⁴J_{HH}=2.4 Hz). ¹³C NMR (75 MHz, CD₂Cl₂): δ 28.8; 31.2; 31.6; 32.8; 35.0; 35.8; 44.9; 124.9; 126.8; 132.9; 140.3; 147.2; 147.6; 180.2. ³¹P NMR (121 MHz, CD₂Cl₂): δ 136.9 (s). ESI-TOF/HRMS: m/e=524.32942 (M+H)⁺. C₃₂H₈NO₃P=523.69; calculated for: C, 73.39; H, 8.85; N, 2.67. Found: C, 73.26; H, 8.74; N, 2.46.

### Example 9

### N-(2,4,8,10-Tetra-tert-butyldibenz[d,f]{1,3,2}dioxaphosphepin-6-yl)-azetidin-2-one

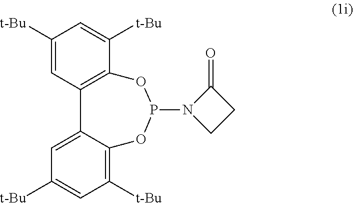 [SMILES: CC(C)(C)c1cc(C(C)(C)C)c2op(N3CCC3=O)oc3c(C(C)(C)C)cc(C(C)(C)C)cc3c2c1]

Yield: 92%; white solid. ¹H NMR (300 MHz, CDCl₃): δ 1.28 (s, 18H), 1.39 (s, 18H), 2.70-2.81 (br, s, 2H), 2.81-2.88 (m, 2H), 7.08 (d, 2H, J=2.4 Hz), 7.37 (d, 2H, J=2.4 Hz). ³¹P NMR (121 MHz, CDCl₃): δ 128.95 (s). ¹³C NMR (75 MHz, CDCl₃): δ 31.0 (d, J=2.4 Hz, (CH₃)₃C), 31.5 (s, (CH₃)₃C), 34.7 (s, (CH₃)₃C), 35.5 (s, (CH₃)₃C), 36.9 (s, CH₂), 37.5 (d, J=7.8 Hz, CH₂), 124.4 (s, CH_{Ar}), 126.5 (s, CH_{Ar}), 132.7 (d, J=3.7 Hz, C_{Ar}), 140.0 (d, J=1.7 Hz, C_{Ar}), 146.4 (d, J=5.1 Hz, C_{Ar}), 147.1 (s, C_{Ar}), 170.7 (d, ²J=20.5 Hz, C═O). HRMS (ESI-TOF/MS): calculated m/z (C₃₁H₄₅N₁O₃P₁, (M+H)⁺) 510.3132; found 510.314; calculated m/z (C₃₁H₄₄N₁Na₁O₃P₁, (M+Na)⁺) 532.2951; found 532.296.

### Example 10

### N-(2,4,8,10-Tetra-tert-butyldibenz[d,f]{1,3,2}dioxaphosphepin-6-yl)-N,N′-dimethylurea

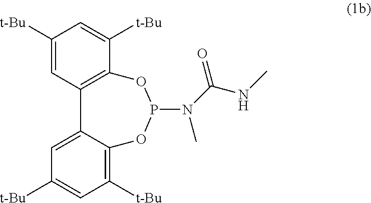 [SMILES: CNC(=O)N(C)p1oc2c(C(C)(C)C)cc(C(C)(C)C)cc2c2cc(C(C)(C)C)cc(C(C)(C)C)c2o1]

Yield: 71%; white solid (recrystallized from CH₃CN/THF (2.4/1)); ¹H NMR (300 MHz, CDCl₃): δ 1.28 (s, 18H), 1.36 (s, 18H), 2.48 (br, s, 3H), 2.83 (br, s, 3H), 5.50 (br, s, 1H), 7.11 (d, 2H, J=2.4 Hz), 7.37 (d, 2H, J=2.4 Hz). ³¹P NMR (121 MHz, CDCl₃): δ 135.4 (br, s, 80% integrated area), 139.3 (br, s, 20% integrated area). The two signals partly overlap. The ratio is solvent-dependent. ³¹P NMR (121 MHz, PhCH₃/CDCl₃=2/1): δ 135.4 (br, s, 88% integrated area), 139.3 (br, s, 12% integrated area). The cause of the appearance of two sets of signals is the occurrence of tautomeric structures. ¹³C NMR (75 MHz, CDCl₃): δ 27.5 (s, CH₃NC(0)), 30.9 (d, J=2.4 Hz, (CH₃)₃C), 31.5 (s, (CH₃)₃C), 34.7 (s, (CH₃)₃C), 35.4 (s, (CH₃)C_), 124.6 (s, CH_{Ar}), 126.5 (s, CH_{Ar}), 132.2 (s, C_{Ar}), 140.0 (s, C_{Ar}), 146.7 (d, J=5.4 Hz, C_{Ar}), 146.9 (s, C_{Ar}). HRMS (EI): calculated m/z (C₃₁H₄₇N₂O₃P₁) 526.33156; found 526.33188; MS (EI, 70 eV): m/z (I, %): 526 (2), 456 (100), 441 (79), 57 (26). Anal. calculated for C₃₁H₄₇N₂O₃P₁: C, 70.69; H, 8.99; N, 5.32; P, 5.88. Found C, 70.48; H, 9.03; N, 5.18; P, 5.85.

### Example 11

General method for the synthesis of Rh(acac)(CO)L from the transition metal precursor.

To a stirred solution of Rh(acac)(CO)₂ (1 mmol) in dried CH₂Cl₂ (8 ml) was added dropwise, within 40 min, a solution of the inventive phosphoramidites (1a)-(1i) (1 mmol) in dried CH₂Cl₂ (8 ml). The solution was stirred at room temperature for 2 h. Subsequently, the solvent was distilled off under reduced pressure and the residue was dried in vacuo for 1 h.

### Example 12

In the process according to the invention, the hydroformylation was preferably conducted in a 200 ml autoclave equipped with pressure-retaining valve, gas flow meter, sparging stirrer and pressure pipette as reaction zone. To minimize the influence of moisture and oxygen, the toluene used as solvent was treated with sodium ketyl and distilled under argon. The mixture of the n-octenes used as substrate was heated at reflux over sodium and distilled under argon for several hours. The transition metal was added as a precursor in the form of [(acac)Rh(COD)](acac=acetylacetonate anion; COD=1,5-cyclooctadiene), dissolved in toluene. The latter was mixed with a solution of the respective ligand in the autoclave under an argon atmosphere. The reactor was heated up under synthesis gas pressure and the unsaturated compounds, especially the olefin, the mixture of olefins, were introduced by means of a pressure-resistant pipette once the reaction temperature had been attained. In this case it is advantageous in the process according to the invention to introduce the unsaturated compounds to be hydroformylated into the reaction zone prior to the addition of the hydrogen- and carbon monoxide-containing gas mixture. This applies especially to unsaturated compounds present in a liquid state at room temperature and standard pressure. In these cases, there is no need to add an external solvent, the solvents being the secondary products formed internally, for example those formed in situ during the reaction from the aldol condensation of the primary aldehyde product.

The reaction was conducted at constant pressure. After the reaction time had elapsed, the autoclave was cooled to room temperature, decompressed while stirring and purged with argon. 1 ml of each reaction mixture was removed immediately after the stirrer had been switched off, diluted with 5 ml of pentane and analysed by gas chromatography. Inventive working examples are compiled in Table 1, in which one entry also relates to the use of the phosphite ligands known by the CAS Registry Numbers [93347-72-9], [31570-04-4]—trade name Alkanox®240.

TABLE 1
Hydroformylation of unfunctionalized olefins^{a}

| | | | k_{obs.} | Yield | Selectivity |
|---|---|---|---|---|---|
| Ligand | Structure | Substrate | [min⁻¹] | [%] | [%] |
| (1f) | 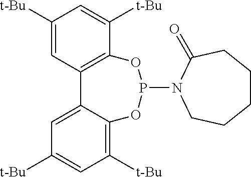 [SMILES: CC(C)(C)c1cc(C(C)(C)C)c2op(N3CCCCCC3=O)oc3c(C(C)(C)C)cc(C(C)(C)C)cc3c2c1] | n- octenes^{b} | n.d.^{c} | 20 | 24.9 |
| (1g) | 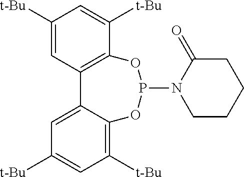 [SMILES: CC(C)(C)c1cc(C(C)(C)C)c2op(N3CCCCC3=O)oc3c(C(C)(C)C)cc(C(C)(C)C)cc3c2c1] | n- octenes^{b} | n.d.^{c} | 32 | 24.0 |
| (1h) | 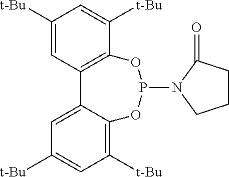 [SMILES: CC(C)(C)c1cc(C(C)(C)C)c2op(N3CCCC3=O)oc3c(C(C)(C)C)cc(C(C)(C)C)cc3c2c1] | n- octenes^{b} | 0.106 | 98 | 18.6 |
| (1i) | 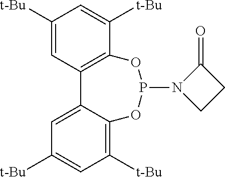 [SMILES: CC(C)(C)c1cc(C(C)(C)C)c2op(N3CCC3=O)oc3c(C(C)(C)C)cc(C(C)(C)C)cc3c2c1] | n- octenes^{b} | 0.1718 | 96 | 21.2 |
| (1d) | 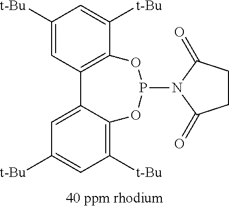 [SMILES: CC(C)(C)c1cc(C(C)(C)C)c2op(N3C(=O)CCC3=O)oc3c(C(C)(C)C)cc(C(C)(C)C)cc3c2c1] | n- octenes^{b} | n.d.^{c} | 93 | 19.5 |
| (1e) | 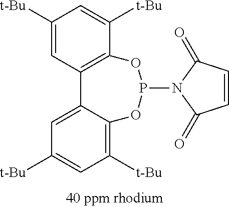 [SMILES: CC(C)(C)c1cc(C(C)(C)C)c2op(N3C(=O)C=CC3=O)oc3c(C(C)(C)C)cc(C(C)(C)C)cc3c2c1] | n- octenes^{b} | 0.038 | 95 | 21.3 |
| (1c) | 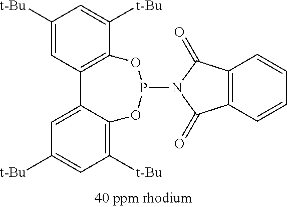 [SMILES: CC(C)(C)c1cc(C(C)(C)C)c2op(N3C(=O)c4ccccc4C3=O)oc3c(C(C)(C)C)cc(C(C)(C)C)cc3c2c1] | n- octenes^{b} | n.d.^{c} | 89 | 15.5 |
| Comparative ligand | Alkanox ®240 as per CAS Reg. No. [93347-72-9], [31570-04-4] | n- octenes | 0.194 | 95 | 20.0 |
| (1a) | 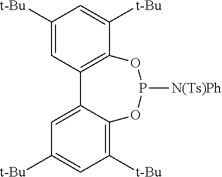 | 2- pentene | n.d.^{c} | 99 | 39.3 |
| (1a) | 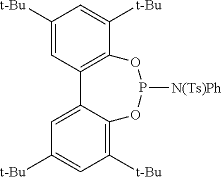 | 1-octene | n.d.^{c} | 96 | 37.0 |

^{a}conditions: P/Rh = 5:1; CO/H₂ = 1:1, 5.0 MPa; 120° C.; toluene;
^{b}consisting of: 1-octene, 3%; cis + trans-2-octene, 49%; cis + trans-3-octene, 29%; cis + trans-octene-4, 16%; structurally isomeric octenes, 3%;
^{c}n.d. = not determinable.

In the rhodium-catalysed hydroformylation of olefins, results achieved with the ligands prepared in accordance with the invention and under conditions selected in accordance with the invention are equally good or even better than with comparable monodentate phosphoramidite and phosphite ligands known from the literature.

### Example 13

### Hydroformylation of Methyl Oleate

[Rh(acac)(CO)₂](1.4 mg, 5.43 μmol) were weighed into a Schlenk vessel under argon and dissolved in toluene (5 ml). 1 ml of this solution was mixed with methyl oleate (1.0 mmol, 0.296 g), ligand (27.5 μmol), octadecane (0.050 g) and toluene (9 ml), and introduced into a 25 ml autoclave. The autoclave was purged three times with nitrogen (10 bar) and once with synthesis gas (CO:H₂=1:1, 1.0 MPa) and then heated up to 80° C. The pressure was adjusted to 2.0 MPa. After a reaction time of 6 h, the autoclave was cooled down. Subsequently, the pressure was released at room temperature and the autoclave was purged twice with nitrogen. Thereafter, a sample was taken for the GC-MS analysis. The solvent was evaporated off from the reaction solution and the yellow oil was analysed by NMR spectroscopy.

Under hydroformylation conditions, the reaction with methyl oleate (MO) as substrate can give not only the desired methyl 9/10-formylstearate (MFS) but also the isomerized olefin (methyl elaidate=ME) and the hydrogenation product (methyl stearate=MS). Table 2 summarizes typical examples.

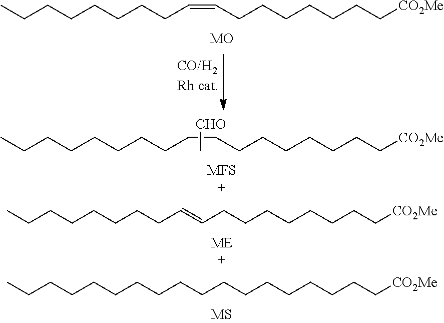

Analysis for Determination of Regioselectivity

For characterization and calibration of the product (MFS), an isomerization-free hydroformylation with triphenylphosphine was used according to the method of Vogl et

al., PhD Thesis, Rostock 2009, as shown below.

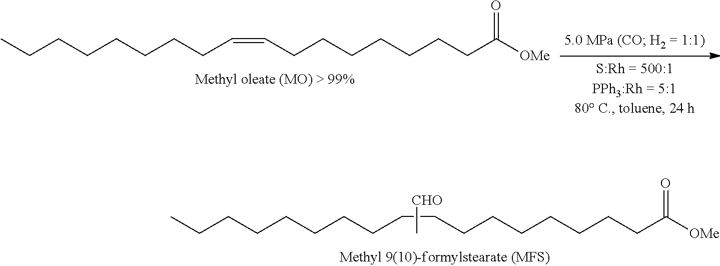

To purify the hydroformylation products of the methyl oleate, the reaction mixture was distilled in a Kugelrohr distillation apparatus (1.5×10⁻¹ mbar/180° C.). The pure methyl formylstearate was used for the calibration, employing octadecane as internal standard. In product purification experiments by means of column chromatography (cyclohexane:ethyl acetate), the formyl product decomposed on the column.

In order to determine the exact position of the aldehyde group, the hydroformylation products were analysed by GC/MS. The fact that the aldehydes are very air-sensitive is known from the prior art; Frankel et al. in J. Am. Oil Chem. Soc. 1969, 46, 133-138 and loc. cit. 1971, 48, 248-253. The aldehydes were oxidized to the corresponding acids and then converted to the methyl esters. The latter were then analysed by GC/MS. The following scheme summarizes the steps

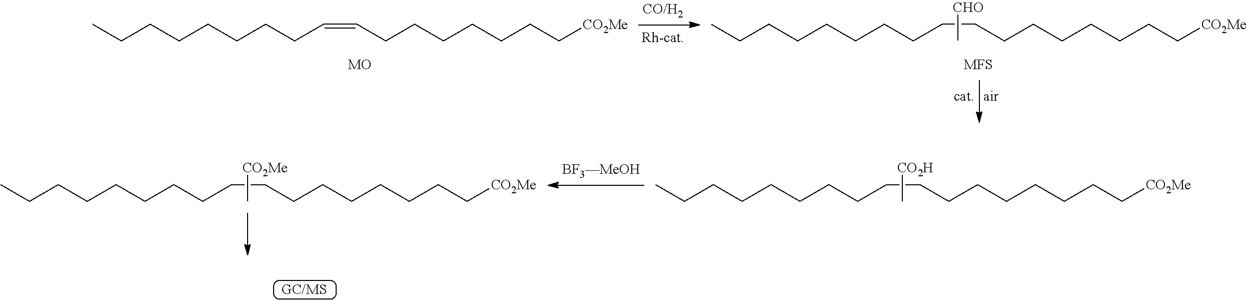

In order to determine the exact position of the aldehyde group, the hydroformylation products were converted to the corresponding methyl esters and analysed by means of GC/MS. The branched formyl products which originate from hydroformylation between carbon atoms 3 and 17 are characterized by the CH3(CH2)ₙCHC(O+.H)OCH3 fragment and, for the linear product (18-MFS), by the CHC(O+.H)OCH3 fragment.

TABLE 2
Hydroformylation of methyl oleate^{a}

| | MO | MFS | ME | MS |
|---|---|---|---|---|
| Ligand | [%] | [%]^{b} | [%] | [%] |
| 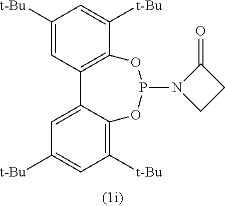 [SMILES: CC(C)(C)c1cc(C(C)(C)C)c2op(N3CCC3=O)oc3c(C(C)(C)C)cc(C(C)(C)C)cc3c2c1] | 0.8 | 92.9 | 5.8 | 0.5 |
| 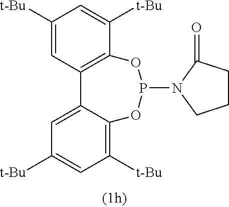 [SMILES: CC(C)(C)c1cc(C(C)(C)C)c2op(N3CCCC3=O)oc3c(C(C)(C)C)cc(C(C)(C)C)cc3c2c1] | 8.5 | 58.1 | 0.5 | 32.9 |
| 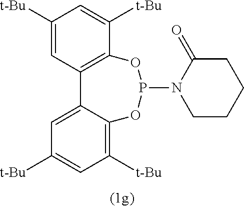 [SMILES: CC(C)(C)c1cc(C(C)(C)C)c2op(N3CCCCC3=O)oc3c(C(C)(C)C)cc(C(C)(C)C)cc3c2c1] | 11.3 | 32.2 | 56.2 | 0.3 |
| 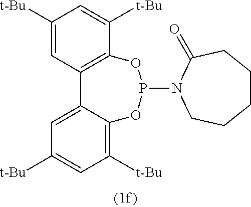 [SMILES: CC(C)(C)c1cc(C(C)(C)C)c2op(N3CCCCCC3=O)oc3c(C(C)(C)C)cc(C(C)(C)C)cc3c2c1] | 1.3 | 89.1 | 9.3 | 0.3 |

^{a}reaction conditions: ligand /Rh = 25/1; substrate. Rh = 910:1; 2.0 MPa (CO:H₂ = 1:1); 80° C., toluene, 6 h;
^{b}always also contains traces of 7/8/11/12-monoformyl esters, but these are not detectable to an exactly quantitative degree.

### Example 14

The influence of the synthesis gas pressure on the hydroformylation with the ligand (1i) is shown in Table 3, the other reaction parameters having been kept constant from Example 13. It is apparent from this that the proportion of desired hydroformylation product increases with rising pressure. Isomerization and hydrogenation are no longer observed at 4.0 and 6.0 MPa.

TABLE 3
Variation of the synthesis gas pressure in the
Rh-catalysed hydroformylation with ligand (1i).

| Pressure [MPa] | Conversion [%] | MFS [%] | MO [%] | ME [%] | MS [%] |
|---|---|---|---|---|---|
| 1.0 | 99.4 | 93.2 | 0.6 | 6.1 | 0.2 |
| 2.0 | 99.2 | 92.9 | 0.8 | 5.8 | 0.5 |
| 4.0 | >99.5 | >99 | — | — | — |
| 6.0 | >99.5 | >99 | — | — | — |

### Example 15

The influence of the temperature on the hydroformylation with the ligand (1i) is shown in Table 4, the other reaction parameters having been kept constant from Example 13. It is apparent from this that the proportion of unwanted isomerization product decreases with rising temperature.

TABLE 4
Variation of the temperature in the Rh-catalysed
hydroformylation with ligand (1i).

| T [° C.] | Conversion [%] | MFS [%] | MO [%] | ME [%] | MS [%] |
|---|---|---|---|---|---|
| 60 | 99 | 89.9 | 1.0 | 9.0 | 0.1 |
| 80 | 99.2 | 92.9 | 0.8 | 5.8 | 0.5 |
| 100 | 99.9 | 98.6 | 0.1 | 1.0 | 0.3 |
| 120 | 99.7 | 95.9 | 0.3 | 2.8 | 1.4 |

The novel ligands are particularly suitable in the regioselective hydroformylation of unsaturated fatty acid derivatives. When Z-olefins are used, the unwanted isomerization to the E-olefins is almost entirely suppressed at elevated temperatures and synthesis gas pressures. The proportion of hydrogenation products is likewise very low.

### Example 16

### Hydrolysis Experiments

To a 0.0175 M solution of the phosphoramidite in dried 1,4-dioxane were added 20 equivalents of distilled water. This sample was divided between two NMR tubes which had been dried under reduced pressure beforehand with a flame and which contained tri-n-octylphosphine oxide in o-xylene-D10 as external standard. For comparison, one sample was stored at room temperature, the other heated to 80-85° C. The samples were analysed quantitatively by means of ³¹P NMR spectroscopy (manually adjusted lock signal based on CDCl₃, NS=256, D1=5 sec).

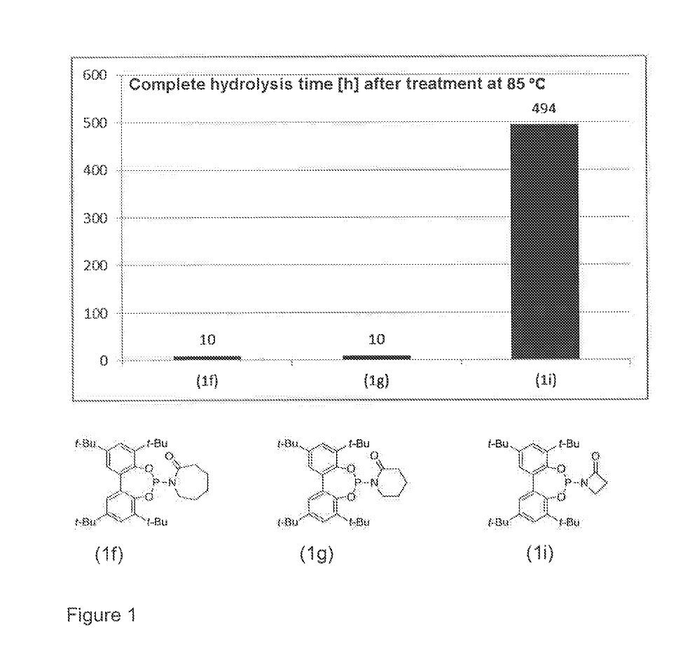

As apparent from FIG. 1, the phosphoramidite (1i) which derives from a 4-membered lactam ring is almost 50 times more stable than those phosphoramidites having larger lactam rings (1f) and (1g).

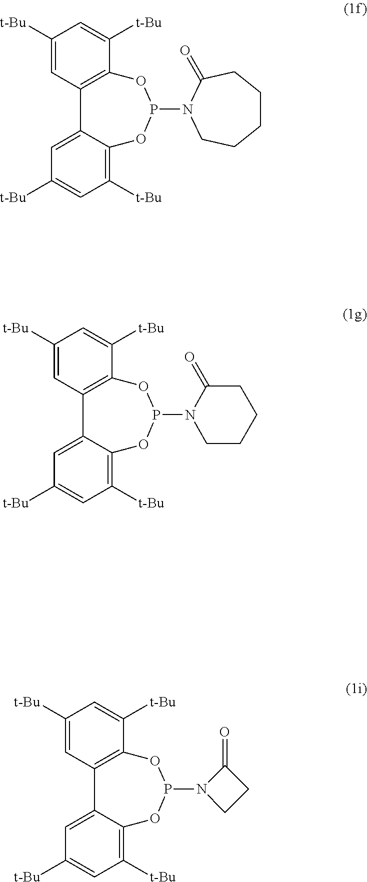 [SMILES: CC(C)(C)c1cc(C(C)(C)C)c2op(N3CCCCCC3=O)oc3c(C(C)(C)C)cc(C(C)(C)C)cc3c2c1 ; CC(C)(C)c1cc(C(C)(C)C)c2op(N3CCCCC3=O)oc3c(C(C)(C)C)cc(C(C)(C)C)cc3c2c1 ; CC(C)(C)c1cc(C(C)(C)C)c2op(N3CCC3=O)oc3c(C(C)(C)C)cc(C(C)(C)C)cc3c2c1]

High hydrolysis stability, in addition to high catalytic activity, is a main criterion for the use of ligands in industrial scale hydroformylation processes.

As already detailed in the prior art and elucidated above—for example in U.S. Pat. No. 5,364,950, U.S. Pat. No. 5,763,677 and also in “Catalyst Separation, Recovery and Recycling”, edited by D. J, Cole-Hamilton, R. P. Tooze, 2006, NL, pages 25-26, and in Rhodium-catalyzed Hydroformylation, ed. by P. W. N. M. van Leeuwen et C. Claver, Kluwer Academic Publishers 2006, AA Dordrecht, NL, pages 206-211—degradation products from the breakdown of the catalytically active composition do not just lead to shortened onstream times of the industrial scale process.

In addition, their existence promotes unwanted further reactions of the target products, the aldehydes, which reduce the yield of target products and hence the overall economic viability of the industrial scale process.

It is found that the phosphoramidite (1i) according to the invention fulfills the task of providing hydrolysis-stable ligands in an outstanding manner.

It is found that the inventive phosphoramidite (1i) achieves the object of providing hydrolysis-stable ligands in an outstanding manner.

1. Phosphoramidites of the formula (I) 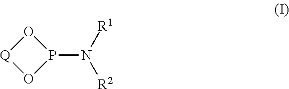
where Q is a divalent substituted or unsubstituted aromatic radical;
where R¹ is not the same as R² and they are independently selected from alkyl, aryl, carboxamide and organosulphonyl radicals;
or R¹ and R² together with N form a heterocyclic structure selected from lactams, dicarboximides.

2. Phosphoramidites according to claim 1, where Q is selected from substituted or unsubstituted 1,1′-biphenyl, 1,1′-binaphthyl and ortho-phenyl radicals.

3. Phosphoramidites according to claim 2, where Q is selected from substituted or unsubstituted 1,1′-biphenyl radicals.

4. Phosphoramidites according to claim 3, where R¹ is not the same as R² and they are independently selected from C₁-C₅-alkyl, aryl, carboxamide and tosyl radicals.

5. Phosphoramidites according to claim 4, where the compounds are selected from:

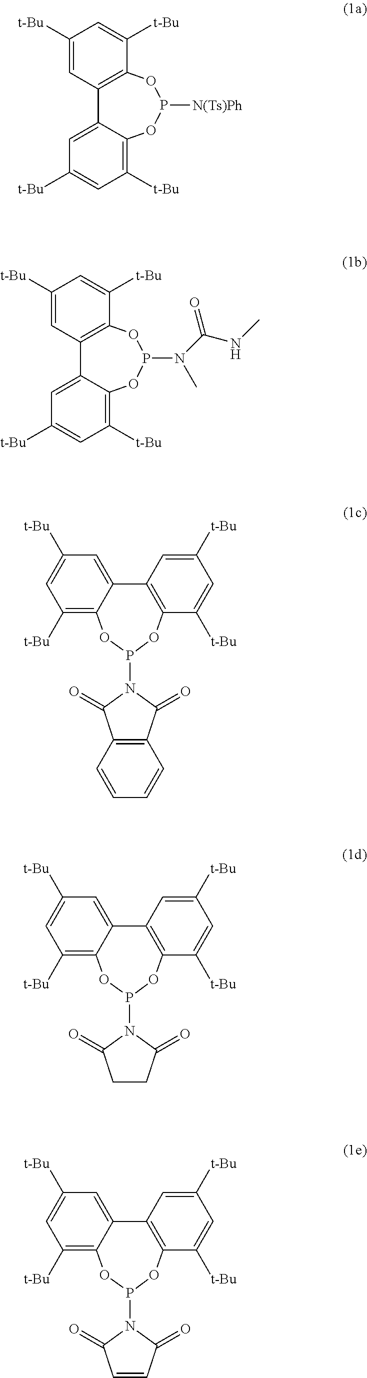

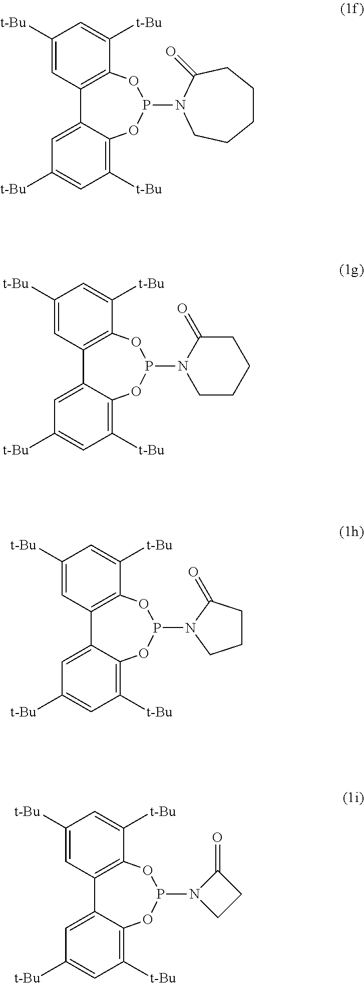 [SMILES: CC(C)(C)c1cc(C(C)(C)C)c2op(N3CCCCCC3=O)oc3c(C(C)(C)C)cc(C(C)(C)C)cc3c2c1 ; CC(C)(C)c1cc(C(C)(C)C)c2op(N3CCCCC3=O)oc3c(C(C)(C)C)cc(C(C)(C)C)cc3c2c1 ; CC(C)(C)c1cc(C(C)(C)C)c2op(N3CCCC3=O)oc3c(C(C)(C)C)cc(C(C)(C)C)cc3c2c1 ; CC(C)(C)c1cc(C(C)(C)C)c2op(N3CCC3=O)oc3c(C(C)(C)C)cc(C(C)(C)C)cc3c2c1]

6. Transition metal compounds of the formula Me(acac)(CO)L with Me=transition metal, where L is selected from: 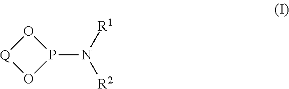
where Q is a divalent substituted or unsubstituted aromatic radical;
where R¹ is not the same as R² and they are independently selected from alkyl, aryl, carboxamide and organosulphonyl radicals;
or R¹ and R² together with N form a heterocyclic structure selected from lactams, dicarboximides.

7. Transition metal compounds according to claim 6, where Q is selected from substituted or unsubstituted 1,1′-biphenyl, 1,1′-binaphthyl and ortho-phenyl radicals.

8. Transition metal compounds according to claim 7, where Q is selected from substituted or unsubstituted 1,1′-biphenyl radicals.

9. Transition metal compounds according to claim 8, where R¹ is not the same as R² and they are independently selected from C₁-C₅-alkyl, aryl, carboxamide and tosyl radicals.

10. Transition metal compounds of the formula Me(acac)(CO)L with Me=transition metal according to claim 9, where L is selected from:

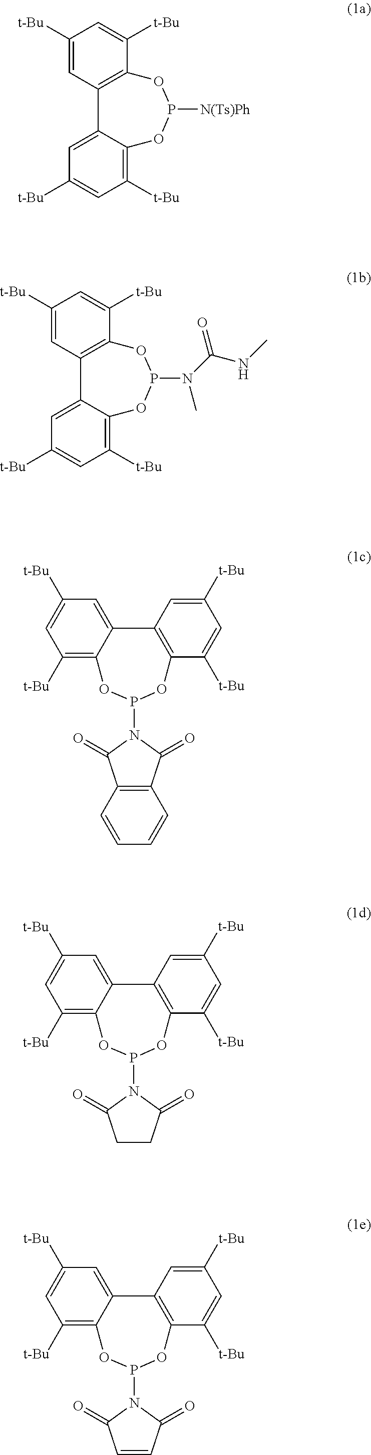

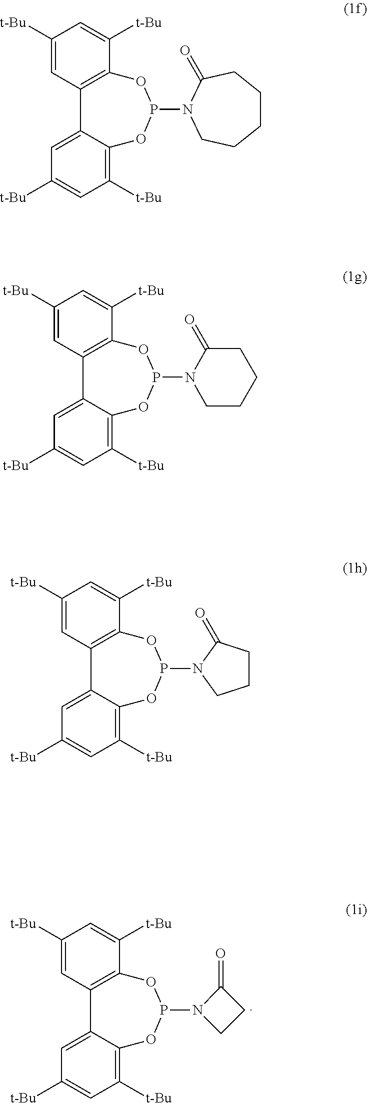 [SMILES: CC(C)(C)c1cc(C(C)(C)C)c2op(N3CCCCCC3=O)oc3c(C(C)(C)C)cc(C(C)(C)C)cc3c2c1 ; CC(C)(C)c1cc(C(C)(C)C)c2op(N3CCCCC3=O)oc3c(C(C)(C)C)cc(C(C)(C)C)cc3c2c1 ; CC(C)(C)c1cc(C(C)(C)C)c2op(N3CCCC3=O)oc3c(C(C)(C)C)cc(C(C)(C)C)cc3c2c1 ; CC(C)(C)c1cc(C(C)(C)C)c2op(N3CCC3=O)oc3c(C(C)(C)C)cc(C(C)(C)C)cc3c2c1]

11. Transition metal compounds of the formula Me(acac)(CO)L with Me=transition metal according to claim 10, where Me is selected from rhodium, iridium, ruthenium, cobalt.

12. Transition metal compounds according to claim 11, where the transition metal is rhodium.

13. Catalytically active compositions in the hydroformylation comprising:
a) transition metal compounds according to claims 6-12;
b) free ligands according to claims 1-5;
c) solvents.

14. Use of a catalytically active composition according to claim 13 in a process for hydroformylating unsaturated compounds.

15. Process for hydroformylating unsaturated compounds using a catalytically active composition according to claim 13, where the unsaturated compounds are selected from:
hydrocarbon mixtures from steamcracking plants;
hydrocarbon mixtures from catalytically operated cracking plants;
hydrocarbon mixtures from oligomerization processes;
hydrocarbon mixtures comprising polyunsaturated compounds;
olefin-containing mixtures including olefins having up to 30 carbon atoms;
unsaturated carboxylic acid derivatives.

16. Process according to claim 15, wherein, in a first process step, phosphoramidites according to claims 1-5 are initially charged as ligands in at least one reaction zone, and reacted with a precursor of the transition metal to give a transition metal compound according to claims 6-12 and finally, after adding free ligands according to claims 1-5, and also solvents and a carbon monoxide- and hydrogen-containing gas mixture, to give a catalytically active composition according to claim 13; in a subsequent step, the unsaturated compounds are added under the reaction conditions to form a polyphasic reaction mixture;
after the end of the reaction, the reaction mixture is separated into aldehydes, alcohols, high boilers, ligands, degradation products of the catalytically active composition.

17. Polyphasic reaction mixture comprising:
unsaturated compounds,
a gas mixture including carbon monoxide, hydrogen;
aldehydes,
catalytically active compositions according to claim 13.
